SEQUENCE LISTING (7 sequences)

SEQ ID NO: 1 - DNA, 1788 bp, Hamster
  misc_feature (156)..(1683): Encodes amino acid sequence for the Hamster Scavenger Receptor Class B-I
gccacctgcagggctactgctgctccggccactgcctgagactcaccttgctggaacgtgagcctcggcttctgtcatctctgtggcctctgtcgcttctgtcgctgtcccccttcagtccctgagccccgcgagcccgggccgcacacgcggacatgggcggcagcgccagggcgcgctgggtggcggtggggctgggcgtcgtggggctgctgtgcgctgtgctcggtgtggttatgatcctcgtgatgccctcgctcatcaaacagcaggtactgaagaatgtccgcatagaccccagcagcctgtcctttgcaatgtggaaggagatccctgtacccttctacttgtccgtctacttcttcgaggtggtcaatcccagcgagatcctaaagggtgagaagccagtagtgcgggagcgtggaccctatgtctacagggaattcagacataaggccaacatcaccttcaatgacaatgatactgtgtcctttgtggagcaccgcagcctccatttccagccggacaggtcccacggctctgagagtgactacattatactgcctaacattctggtcttggggggcgcagtaatgatggagagcaagtctgcaggcctgaagctgatgatgaccttggggctggccaccttgggccagcgtgcctttatgaaccgaacagttggtgagatcctgtggggctatgaggatcccttcgtgaattttatcaacaaatacttaccagacatgttccccatcaagggcaagttcggcctgtttgttgagatgaacaactcagactctgggctcttcactgtgttcacgggcgtccagaacttcagcaagatccacctggtggacagatggaatgggctcagcaaggtcaactactggcattcagagcagtgcaacatgatcaatggcacttccgggcagatgtgggcaccattcatgacaccccagtcctcgctggaattcttcagtccggaagcctgcaggtctatgaagctcacctaccatgattcaggggtgtttgaaggcatccccacctatcgcttcacagcccctaaaactttgtttgccaatgggtctgtttacccacccaatgaaggtttctgcccgtgccttgaatccggcattcaaaatgtcagcacttgcaggtttggtgcacccctgtttctgtcacaccctcacttctacaatgcagaccctgtgctatcagaagccgttctgggtctgaaccctgacccaagggagcattctttgttccttgacatccatccggtcactgggatccccatgaactgttctgtgaagttgcagataagcctctacatcaaagctgtcaagggcattgggcaaacagggaagatcgagcccgtggtcctcccattgctgtggtttgagcagagcggtgccatgggcggcgagcccctgaacacgttctacacgcagctggtgctgatgccccaggtacttcagtatgtgcagtatgtgctgctggggctgggcggcctcctgctgctggtgcccgtcatctaccagttgcgcagccaggagaaatgctttttattttggagtggtagtaaaaagggctcgcaggataaggaggccattcaggcctactctgagtctctgatgtcaccagctgccaagggcacggtgctgcaagaagccaagctgtagggtcccaaagacaccacgagcccccccaacctgatagcttggtcagaccagccatccagcccctacaccccgcttcttgaggactctctcagcggacagtcgc

SEQ ID NO: 2 - protein, 509 aa, Hamster
  TRANSMEM (9)..(32): Putative
MGGSARARWVAVGLGVVGLLCAVLGVVMILVMPSLIKQQVLKNVRIDPSSLSFAMWKEIPVPFYLSVYFFEVVNPSEILKGEKPVVRERGPYVYREFRHKANITFNDNDTVSFVEHRSLHFQPDRSHGSESDYIILPNILVLGGAVMMESKSAGLKLMMTLGLATLGQRAFMNRTVGEILWGYEDPFVNFINKYLPDMFPIKGKFGLFVEMNNSDSGLFTVFTGVQNFSKIHLVDRWNGLSKVNYWHSEQCNMINGTSGQMWAPFMTPQSSLEFFSPEACRSMKLTYHDSGVFEGIPTYRFTAPKTLFANGSVYPPNEGFCPCLESGIQNVSTCRFGAPLFLSHPHFYNADPVLSEAVLGLNPDPREHSLFLDIHPVTGIPMNCSVKLQISLYIKAVKGIGQTGKIEPVVLPLLWFEQSGAMGGEPLNTFYTQLVLMPQVLQYVQYVLLGLGGLLLLVPVIYQLRSQEKCFLFWSGSKKGSQDKEAIQAYSESLMSPAAKGTVLQEAKL

SEQ ID NO: 3 - DNA, 1785 bp, Mouse
  misc_feature (51)..(1577): Encodes the amino acid sequence for the murine Scavenger Receptor Class BI
ccgtctccttcaggtcctgagccccgagagccccttccgcgcacgcggacatgggcggcagctccagggcgcgctgggtggccttggggttgggcgccctggggctgctgtttgctgcgctcggcgttgtcatgatcctcatggtgccctccctcatcaagcagcaggtgctcaagaatgtccgcatagacccgagcagcctgtccttcgggatgtggaaggagatccccgtccctttctacttgtctgtctacttcttcgaagtggtcaacccaaacgaggtcctcaacggccagaagccagtagtccgggagcgtggaccctatgtctacagggagttcagacaaaaggtcaacatcaccttcaatgacaacgacaccgtgtccttcgtggagaaccgcagcctccatttccagcctgacaagtcgcatggctcagagagtgactacattgtactgcctaacatcttggtcctggggggctcgatattgatggagagcaagcctgtgagcctgaagctgatgatgaccttggcgctggtcaccatgggccagcgtgcttttatgaaccgcacagttggtgagatcctgtggggctatgacgatcccttcgtgcattttctcaacacgtacctcccagacatgcttcccataaagggcaaatttggcctgtttgttgggatgaacaactcgaattctggggtcttcactgtcttcacgggcgtccagaatttcagcaggatccatctggtggacaaatggaacggactcagcaagatcgattattggcattcagagcagtgtaacatgatcaatgggacttccgggcagatgtgggcacccttcatgacacccgaatcctcgctggaattcttcagcccggaggcatgcaggtccatgaagctgacctacaacgaatcaagggtgtttgaaggcattcccacgtatcgcttcacggcccccgatactctgtttgccaacgggtccgtctacccacccaacgaaggcttctgcccatgccgagagtctggcattcagaatgtcagcacctgcaggtttggtgcgcctctgtttctctcccacccccacttttacaacgccgaccctgtgttgtcagaagctgttcttggtctgaaccctaacccaaaggagcattccttgttcctagacatccatccggtcactgggatccccatgaactgttctgtgaagatgcagctgagcctctacatcaaatctgtcaagggcatcgggcaaacagggaagatcgagccagtagttctgccgttgctgtggttcgaacagagcggagcaatgggtggcaagcccctgagcacgttctacacgcagctggtgctgatgccccaggttcttcactacgcgcagtatgtgctgctggggcttggaggcctcctgttgctggtgcccatcatctgccaactgcgcagccaggagaaatgctttttgttttggagtggtagtaaaaagggctcccaggataaggaggccattcaggcctactctgagtccctgatgtcaccagctgccaagggcacggtgctgcaagaagccaagctatagggtcctgaagacactataagccccccaaacctgatagcttggtcagaccagccacccagtccctacaccccgcttcttgaggactctctcagcggacagcccaccagtgccatggcctgagcccccagatgtcacacctgtccgcacgcacggcacatggatgcccacgcatgtgcaaaaacaactcagggaccagggacagacc

SEQ ID NO: 4 - protein, 509 aa, Mouse
MGGSSRARWVALGLGALGLLFAALGVVMILMVPSLIKQQVLKNVRIDPSSLSFGMWKEIPVPFYLSVYFFEVVNPNEVLNGQKPVVRERGPYVYREFRQKVNITFNDNDTVSFVENRSLHFQPDKSHGSESDYIVLPNILVLGGSILMESKPVSLKLMMTLALVTMGQRAFMNRTVGEILWGYDDPFVHFLNTYLPDMLPIKGKFGLFVGMNNSNSGVFTVFTGVQNFSRIHLVDKWNGLSKIDYWHSEQCNMINGTSGQMWAPFMTPESSLEFFSPEACRSMKLTYNESRVFEGIPTYRFTAPDTLFANGSVYPPNEGFCPCRESGIQNVSTCRFGAPLFLSHPHFYNADPVLSEAVLGLNPNPKEHSLFLDIHPVTGIPMNCSVKMQLSLYIKSVKGIGQTGKIEPVVLPLLWFEQSGAMGGKPLSTFYTQLVLMPQVLHYAQYVLLGLGGLLLLVPIICQLRSQEKCFLFWSGSKKGSQDKEAIQAYSESLMSPAAKGTVLQEAKL

SEQ ID NO: 5 - DNA, 26 bp, Artificial Sequence
  Description of Artificial Sequence primer
tgaaggtggtcttcaagagcagtcct

SEQ ID NO: 6 - DNA, 26 bp, Artificial Sequence
  Description of Artificial Sequence primer
gattgggaagacaatagcaggcatgc

SEQ ID NO: 7 - DNA, 25 bp, Artificial Sequence
  Description of Artificial Sequence primer
tatcctcggcagacctgagtcgtgt

(19) United States
(12) Patent Application Publication
(10) Pub. No.: US 2002/0099040 A1
(43) Pub. Date: Jul. 25, 2002

(54) SR-BI ANTAGONISTS AND USE THEREOF AS CONTRACEPTIVES AND IN THE TREATMENT OF STEROIDAL OVERPRODUCTION

(76) Inventor: MONTY KRIEGER, NEEDHAM, MA (US)

Correspondence Address: Patrea L Pabst, HOLLAND & KNIGHT LLP, One Atlantic Center, 1201 West Peachtree Street Suite 2000, Atlanta, GA, 30309, US

(21) Appl. No.: 09/148,012

(22) Filed: Sep. 4, 1998

Related U.S. Application Data
(60) Provisional application No. 60/057,943, filed on Sep. 5, 1997.

(51) Int. Cl. A61K 31/56; A01N 37/18; A01N 45/00; A61K 38/00

(52) U.S. Cl. 514/169; 514/177; 514/182

(57) ABSTRACT

SR-BI is present on the membranes of hepatocytes and steroidogenic tissues, including the adrenal gland, testes, and ovaries, where it mediates the uptake and transport of cholesteryl ester from high density lipoproteins. It has been demonstrated that transgenic animals which do not produce SR-BI are perfectly healthy, with the exception that the females are infertile. This provides evidence that inhibition of uptake, binding or transport of cholesteryl ester to SR-BI can be used to inhibit pregnancy. The same pathway can also be used to decrease production of steroids, and therefore be used as a therapy for disorders involving steroidal overproduction.

### CROSS REFERENCE TO RELATED APPLICATION

This application claims benefit of U.S. Provisional Application No. 60/057,943 filed Sep. 5, 1997.

### STATEMENT REGARDING FEDERALLY SPONSORED RESEARCH

The U.S. government has certain rights to this invention by virtue of Grants HL41484, HI-52212, and HL20948 from the National Institutes of Health-National Heart, Lung and Blood Institute.

### BACKGROUND OF THE INVENTION

The present invention is generally in the area of the prevention of pregnancy and treatment of disorders involving steroidal overproduction, such as Cushings' disease, or disorders which can be treated by lowering steroid levels, such as endometriosis and breast and prostate cancer, by inhibition of binding and uptake of cholesterol and other lipids via the SR-BI scavenger receptor.

The intercellular transport of lipids through the circulatory system requires the packaging of these hydrophobic molecules into water-soluble carriers, called lipoproteins, and the regulated targeting of these lipoproteins to appropriate tissues by receptor-mediated pathways. The most well characterized lipoprotein receptor is the LDL receptor, which binds to apolipoproteins B-100 (apoB-100) and E (apoE), which are constituents of low density lipoprotein (LDL), the principal cholesteryl-ester transporter in human plasma, very low-density lipoprotein (VLDL), a triglyceride-rich carrier synthesized by the liver, intermediate-density lipoprotein (IDL), and catabolized chylomicrons (dietary triglyceride-rich carriers).

All members of the LDL receptor gene family consist of the same basic structural motifs. Ligand-binding (complement-type) cysteine-rich repeats of approximately 40 amino acids are arranged in clusters (ligand-binding domains) that contain between two and eleven repeats. Ligand-binding domains are always followed by EGF-precursor homologous domains. In these domains, two EGF-like repeats are separated from a third EGF-repeat by a spacer region containing the YWTD motif. In LRP and gp330, EGF-precursor homologous domains are either followed by another ligand-binding domain or by a spacer region. The EGF-precursor homology domain, which precedes the plasma membrane, is separated from the single membrane-spanning segment either by an O-linked sugar domain (in the LDL receptor and VLDL receptor) or by one (in C. elegans and gp330) or six EGF-repeats (in LRP). The cytoplasmic tails contain between one and three “NPXY” internalization signals required for clustering of the receptors in coated pits. In a later compartment of the secretory pathway, LRP is cleaved within the eighth EGF-precursor homology domain. The two subunits LRP-515 and LRP-85 (indicated by the brackets) remain tightly and non-covalently associated. Only partial amino acid sequence of the vitellogenin receptor and of gp330 are available.

LDL receptors and most other mammalian cell-surface receptors that mediate binding and, in some cases, the endocytosis, adhesion, or signaling exhibit two common ligand-binding characteristics: high affinity and narrow specificity. However, two additional lipoprotein receptors have been identified which are characterized by high affinity and broad specificity: the macrophage scavenger receptors class A type I and type II.

Scavenger receptors mediate the endocytosis of chemically modified lipoproteins, such as acetylated LDL (AcLDL) and oxidized LDL (OxLDL), and have been implicated in the pathogenesis of atherosclerosis (Krieger and Herz, 1994 Annu. Rev. Biochem. 63, 601-637; Brown and Goldstein, 1983 Annu. Rev. Biochem. 52, 223-261; Steinberg et al., 1989 N. Engl. J. Med. 320, 915-924). Macrophage scavenger receptors exhibit complex binding properties, including inhibition by a wide variety of polyanions, such as maleylated BSA (M-BSA) and certain polynucleotides and polysaccharides, as well as unusual ligand-cross competition (Freeman et al., 1991 Proc. Natl. Acad. Sci. U.S.A. 88, 4931-4935, Krieger and Herz, 1994). Several investigators have suggested that there may be at least three different classes of such receptors expressed on mammalian macrophages, including receptors which recognize either AcLDL or OxLDL, or both of these ligands (Sparrow et al., 1989 J. Biol. Chem. 264, 2599-2604; Arai et al., 1989 Biochem. Biophys. Res. Commun. 159, 1375-1382; Nagelkerke et al., 1983 J. Biol. Chem. 258, 12221-12227).

The first macrophage scavenger receptors to be purified and cloned were the mammalian class A type I and II receptors. These are trimeric integral membrane glycoproteins whose extracellular domains have been predicted to include α-helical coiled-coil, collagenous and globular structures (Kodama et al., 1990 Nature 343, 531-535; Rohrer et al., 1990 Nature 343, 570-572; Krieger and Herz, 1994). The collagenous domain, shared by the class A type I and type II receptors, apparently mediates the binding of polyanionic ligands (Acton et al., 1993 J. Biol. Chem. 268, 3530-3537; Doi et al., 1993 J. Biol. Chem. 268, 2126-2133). The class A type I and type II molecules, which are the products of alternative splicing of a single gene, are hereafter designated class A scavenger receptors (SR-AI and SR-AII). The class A receptors, which bind both AcLDL and OxLDL (Freeman et al., 1991), have been proposed to be involved in host defense and cell adhesion, as well as atherogenesis (Freeman et al., 1991; Krieger, 1992 Trends Biochem. Sci. 17, 141-146; Fraser et al., 1993 Nature 364, 343-346; Krieger and Herz, 1994).

Based on models of the predicted quaternary structures of the class A type I and type II macrophage scavenger receptors, both contain six domains, of which the first five are identical: the N-terminal cytoplasmic region, the transmembrane region, spacer, α-helical coil, and collagen-like domains. The C-terminal sixth domain of the type I receptor is composed of an eight-residue spacer followed by a 102-amino acid cysteine-rich domain (SRCR), while the sixth domain of the type II receptor is only a short oligopeptide.

Using a murine macrophage cDNA library and a COS cell expression cloning technique, Endemann, Stanton and colleagues, (Endemann, et al. 1993 J. Biol. Chem. 268, 11811-11816; Stanton, et al. J. Biol. Chem. 267, 22446-22451), reported the cloning of cDNAs encoding two additional proteins that can bind OxLDL. The binding of OxLDL to these proteins was not inhibited by AcLDL. These proteins are FcgRII-B2 (an Fc receptor) (Stanton et al., 1992) and CD36 (Endemann et al., 1993). The significance of the binding of OxLDL to FcgRII-B2 in transfected COS cells is unclear because FcgRII-B2 in macrophages apparently does not contribute significantly to OXLDL binding (Stanton et al., 1992). However, CD36 may play a quantitatively significant role in OxLDL binding by macrophages (Endemann et al., 1993). In addition to binding oxidized LDL, CD36 binds thrombospondin (Asch et al., 1987 J. Clin. Invest. 79, 1054-1061), collagen (Tandon et al., 1989 J. Biol. Chem. 264, 7576-7583), long-chain fatty acids (Aburnrad et al., 1993 J. Biol. Chem. 268, 17665-17668) and Plasmodium falciparum infected erythrocytes (Oquendo et al., 1989 Cell 58, 95-101). CD36 is expressed in a variety of tissues, including adipose, and in macrophages, epithelial cells, monocytes, endothelial cells, platelets, and a wide variety of cultured lines (Abumrad et al., 1993; and see Greenwalt et al., 1992 Blood 80, 1105-1115 for review). Although the physiologic functions of CD36 are not known, it may serve as an adhesion molecule due to its collagen-binding properties. It is also been proposed to be a long-chain fatty acid transporter (Abumrad et al., 1993) and a signal transduction molecule (Ockenhouse et al., 1989 J. Clin. Invest. 84, 468-475; Huang et al., 1991 Proc. Natl. Acad. Sci. USA 88, 7844-7848), and may serve as a receptor on macrophages for senescent neutrophils (Savill et al., 1991 Chest 99, 7 (suppl)).

Modified lipoprotein scavenger receptor activity has also been observed in endothelial cells (Arai et al., 1989; Nagelkerke et al., 1983; Brown and Goldstein, 1983; Goldstein et al., 1979 Proc. Natl. Acad. Sci. U.S.A. 76, 333-337). At least some of the endothelial cell activity apparently is not mediated by the class A scavenger receptors (Bickel et al., 1992 J. Clin. Invest. 90, 1450-1457; Arai et al., 1989; Nagelkerke et al., 1983; Via et al., 1992 The Faseb J. 6, A371), which are often expressed by macrophages (Naito et al., 1991 Am. J. Pathol. 139, 1411-1423; Krieger and Herz, 1994). In vivo and in vitro studies suggest that there may be scavenger receptor genes expressed in endothelial cells and macrophages which differ from both the class A scavenger receptors and CD36 (Haberland et al., 1986 J. Clin. Inves. 77, 681-689; Via et al., 1992; Sparrow et al., 1989; Horiuchi et al., 1985 J. Biol. Chem. 259, 53-56; Arai et al., 1989; and see below). Via, Dressel and colleagues (Ottnad et al., 1992 Biochem J. 281, 745-751) and Schnitzer et al. 1992 J. Biol. Chem. 267, 24544-24553) have detected scavenger receptor-like binding by relatively small membrane associated proteins of 15-86 kD. In addition, the LDL receptor related protein (LRP) has been shown to bind lipoprotein remnant particles and a wide variety of other macromolecules. Both the mRNA encoding LRP and the LRP protein are found in many tissues and cell types (Herz, et al., 1988 EMBO J. 7:4119-4127; Moestrup, et al., 1992 Cell Tissue Res. 269:375-382), primarily the liver, the brain and the placenta. The predicted protein sequence of the LRP consists of a series of distinctive domains or structural motifs, which are also found in the LDL receptor.

As described by Kreiger, et al., in PCT/US95/07721 “Class BI and CI Scavenger Receptors” Massachusetts Institute of Technology (“Krieger, et al.”), two distinct scavenger receptor type proteins having high affinity for modified lipoproteins and other ligands have been isolated, characterized and cloned. Hamster and murine homologs of SR-BI, an AcLDL and LDL binding scavenger receptor, which is distinct from the class A type I and type II macrophage scavenger receptors, has been isolated and characterized. In addition, DNA encoding the receptor cloned from a variant of Chinese Hamster Ovary Cells, designated Var-261, has been isolated and cloned. dSR-CI, a non-mammalian AcLDL binding scavenger receptor having high ligand affinity and broad specificity, was isolated from Drosophila melanogaster.

It was reported by Kreiger, et al. that the SR-BI receptor is expressed principally in steroidogenic tissues and liver and appears to mediate HDL-transfer and uptake of cholesterol. Competitive binding studies show that SR-BI binds LDL, modified LDL, negatively charged phospholipid, and HDL. Direct binding studies show that SR-BI expressed in mammalian cells (for example, a varient of CHO cells) binds HDL, without cellular degradation of the HDL-apoprotein, and lipid is accumulated within cells expressing the receptor. These studies indicate that SR-BI might play a major role in transfer of cholesterol from peripheral tissues, via HDL, into the liver and steroidogenic tissues, and that increased or decreased expression in the liver or other tissues may be useful in regulating uptake of cholesterol by cells expressing SR-BI, thereby decreasing levels in foam cells and deposition at sites involved in atherogenesis.

Atherosclerosis is the leading cause of death in western industrialized countries. The risk of developing atherosclerosis is directly related to plasma levels of LDL cholesterol and inversely related to HDL cholesterol levels. Over 20 years ago, the pivotal role of the LDL receptor in LDL metabolism was elucidated by Goldstein, et al., in the Metabolic and Molecular Bases of Inherited Disease, Scriver, et al. (McGraw-Hill, NY 1995), pp. 1981-2030. In contrast, the cellular mechanisms responsible for HDL metabolism are still not well defined. It is generally accepted that HDL is involved in the transport of cholesterol from extrahepatic tissues to the liver, a process known as reverse cholesterol transport, as described by Pieters, et al., Biochim. Biophys. Acta 1225, 125 (1994), and mediates the transport of cholesteryl ester to steroidogenic tissues for hormone synthesis, as described by Andersen and Dietschy, J. Biol. Chem. 256, 7362 (1981). The mechanism by which HDL cholesterol is delivered to target cells differs from that of LDL. The receptor-mediated metabolism of LDL has been thoroughly described and involves cellular uptake and degradation of the entire particle. In contrast, the receptor-mediated HDL metabolism has not been understood as well. Unlike LDL, the protein components of HDL are not degraded in the process of transporting cholesterol to cells. Despite numerous attempts by many investigators, the cell-surface protein(s) that participate in the delivery of cholesterol from HDL to cells had not been identified before the discovery that SR-BI was an HDL receptor.

It is an object of the present invention to provide methods and reagents for designing drugs that can stimulate or inhibit the binding to and lipid movements mediated by SR-BI and redirect uptake and metabolism of lipids and cholesterol by cells.

### SUMMARY OF THE INVENTION

SR-BI is present at relatively high levels on the membranes of hepatocytes and steroidogenic tissues, including the adrenal gland, testes, and ovaries, where it mediates the uptake and transport of cholesteryl ester from high density lipoproteins. It has been demonstrated that transgenic animals which do not produce SR-BI are healthy, with the exception that the females are infertile. This provides evidence that inhibition of uptake, binding or transport of cholesteryl ester to SR-BI can be used to inhibit pregnancy. The same pathway can also be used to decrease production of steroids, and therefore be used as a therapy for disorders involving steroidal overproduction and disorders treated with drugs that decrease steroids, such as endometriosis, and breast and prostate cancer.

Methods for regulation of cholesterol transport are described which are based on regulation of the expression or function of the SR-BI HDL receptor. The examples demonstrate that estrogen dramatically downregulates hepatic SR-BI under conditions of tremendous upregulation of the LDL-receptor. The examples also demonstrate the upregulation of SR-BI in rat adrenal membranes and other non-placental steroidogenic tissues from animals treated with estrogen, but not in other non-placental non-steroidogenic tissues, including lung, liver, and skin. Examples further demonstrate that female animals which do not express SR-BI have dramatically reduced levels of offspring, even though they are otherwise healthy and the males normal. Studies demonstrate that they do not produce viable eggs and have a defect involving implantation of normal eggs. Anti-mSR-BI IgG inhibits HDL CE-selective uptake by 70% and cell association of HDL particles by 50% in a dose-dependent manner. The secretion of [³H]steroids derived from HDL containing [³H]CE was inhibited by 78% by anti-mSR-BI IgG.

### BRIEF DESCRIPTION OF THE DRAWINGS

FIGS. 1A-D are graphs of fast pressure liquid chromatography (FPLC) analysis of plasma showing the lipoprotein profile of control (Ad.ΔE1) (FIGS. 1A and 1C) and transgenic mice (Ad.SR-BI) (FIGS. 1B and 1D), and cholesterol levels (micrograms/fraction) over the course of zero to three days (FIGS. 1A and 1B) and seven to twenty-one days (Figures 1C and 1D).

FIG. 2 is a graph of HDL turnover over time (hours) in untreated, normal mice (closed squares), control (AdΔAE1) (open squares) and transgenic mice (Ad.SR-BI) (closed triangles).

FIG. 3 is a schematic of the strategy for targeted disruption of the SR-BI locus in the mouse.

FIG. 4 is the FPLC profiles of plasma lipoprotein cholesterol (A) and apolipoproteins (B) for wild-type (srbI^{+/+}) and heterozygous (srbI^{+/+}) and homozygous (srbI^{-/-}) mutant F2 male mice. The chromatograms represent single analyses of pooled samples (150 μl of plasma from 3 animals per sample) from 4-8 h fasted wild-type (srbI^{+/+}, open squares), and heterozygous (srbI^{+/-}, partly filled squares) and homozygous (srbI^{-/-}, filled squares) mutant mice and are representative of multiple, independent determinations. Approximate positions of VLDL, IDL/LDL and HDL elution are indicated by brackets and were determined both by analysis of human lipoprotein standards and by previous analysis of lipoproteins in murine plasma.

FIGS. 5A and 5B are graphs of the effects of 356 anti-mSR-BI IgG on DiI uptake from diI HDL by ldlA[mSR-BI] cells. FIG. 5A is a graph of ldlA[mSR-BI] cells incubated for 2 hr with DiI-HDL (10 μg protein/ml) in medium containing the indicated concentration of 356 anti-mSR-BI

FIGS. 6A and 6B show the selective CE uptake and cell association of [125I,3H]hHDL3 by Y1-BS1 cells. Y1-BS1 cells were incubated with the indicated concentrations of [125I,3H]hHDL3 for 4 hr, after which the cells were processed to determine selective CE uptake (FIG. 6A) and cell association of HDL apolipoprotein (FIG. 6B). The high-affinity (▴) component for each of these parameters was resolved from the total measured value (●) as described. Error bars represent the range of duplicate determinations.

FIGS. 7A-7C are graphs of the effects of 356 anti-mSR-BI IgG on HDL-selective CE uptake and HDL cell association. Y1-BS1 cells were incubated for 2 hr with [¹²⁵I,3H]hHDL3 (10 μg protein/ml) in medium containing the indicated concentration of 356 anti-mSR-BI IgG and complementary amounts of nonimmune IgG to give a final IgG concentration of 6 mg/ml. Cells were processed to determine HDL-selective CE uptake (FIG. 7A) and cell association of HDL apolipoprotein (FIG. 7B). The 100% of control value in each case refers to samples incubated with 6 mg/ml nonimmune IgG. (FIGS. 7A and B) Results for 0.0 mg/ml and 6.0 mg/ml anti-mSR-BI IgG are the means of 22 samples (±SEM) from seven experiments. The results for the intermediate anti-mSR-BI IgG concentrations are the means of eight samples (±SEM) from four experiments. (FIG. 7C) HDL-selective CE uptake (open bars) and cell-associated HDL apolipoprotein (stippled bars) in the presence of no IgG (100% of control value) in comparison with cells incubated with 6 mg/ml nonimmune IgG or with excess unlabeled HDL (500 μg protein/ml). For the no IgG samples, the results are the means of 20 samples (±SEM) from seven experiments. The results for the 6 mg/ml NI (nonimmune) IgG are the means of 22 samples (±SEM) from seven experiments, and the 500 μg/ml cold HDL results are the means of 10 samples (±SEM) from 4 experiments.

FIGS. 8A and 8B are graphs of the secretion of [³H] steroid by 1-24ACTH-stimulated Y1-BS1 cells incubated with [³H]hHDL3. Y1-BS1 cells were incubated for 24 hr with 25 μg protein/ml [³H]hHDL3 in the presence or absence of 1 mM aminogluthethimide. S teroids were extracted from the medium with CH2Cl2 and separated by HPLC. FIG. 8A and FIG. 8B are the absorbance profile at 240 nm and the radioactivity profile, respectively. Arrows in FIG. 8A indicate the elution position of standards: corticosterone (I), 11-hydroxyprogesterone (II), 20-hydroxyprogesterone (III), and progesterone (IV).

### DETAILED DESCRIPTION OF THE INVENTION

The role of SR-BI has now been confirmed as the principle mediator of cholesteryl ester transport from peripheral tissues to the liver and other steroidogenic tissues, including the adrenal gland, testes and ovaries.

In previous studies, Western blotting was used to show that upon estrogen treatment in rats levels of SR-BI protein drop dramatically and LDL receptor levels increase in liver. As used herein, steroidogenic tissues refer to non-placental steroidogenic tissues including adrenal, ovary and testes. The liver and non-hepatic steroidogenic tissues had previously been shown to be sites of selective cholesterol uptake from HDL. Fluorescently labeled HDL has been used as a marker of lipid uptake and injected into estrogen and control treated animals. Animals receiving estrogen had significantly reduced levels of SR-BI expressed in the liver, and elevated levels of SR-BI and fluorescence in the ovaries. Since administration of estrogen is associated with a number of side effects, inhibition is more preferably achieved through the use of agents which inhibit expression of SR-BI, translation of SR-BI, binding of SR-BI, or cellular processing mediated by the SR-BI. Inhibition can be direct or indirect, competitive or irreversible. Inhibition of SR-BI can thereby be used to limit steroid production in steroidogenic tissues, and serve either as a means of contraception or a means of treating disorders associated with overproduction of steroids.

I. Inhibitors of SR-BI Transport of Cholesterol.

Direct inhibitors include nucleotide molecules such as antisense oligonucleotides, ribozymes, and triplex forming oligonucleotides which bind to the SR-BI gene, either the protein encoding region of the gene or the regulatory regions of the gene; small organic molecules which bind to the SR-BI protein; soluble SR-BI protein or fragments thereof which competitively bind to the substrate for cell bound SR-BI; and compounds which block binding of HDL to SR-BI.

In a preferred embodiment, these compounds are initially screened using an assay such as the assays described below and then tested in transgenic animals made using standard transgenic animal technology to knockout or overexpress the SR-BI gene. A technique such as embryonic stem cell technology using rats, mice or hamsters or the use of retroviral or adenoviral vectors is preferred, to yield animals expressing some SR-BI.

The cDNA encoding SR-BI has been cloned and is reported in Krieger, et al. The cDNA encoding SR-BI yields a predicted protein sequence of 509 amino acids which is approximately 30% identical to those of the three previously identified CD36 family members. The cloned hamster SR-BI cDNA is approximately 2.9 kb long. The sequences of the 5′ untranslated region, the coding region, and a portion of the 3′ untranslated region are shown in SEQ ID NO: 1. The predicted protein sequence is 509 amino acids (SEQ ID NO:2) with a calculated molecular weight of 57 kD. The murine cDNA is shown in SEQ ID NO: 3 and the predicted amino acid sequence is shown in SEQ ID NO:4.

As used herein, unless specifically stated otherwise, the term “SR-BI” refers to the nucleotide and amino acid sequences, respectively, shown in SEQ ID NOs:1 and 2, and 3 and 4, and degenerate variants thereof and their equivalents in other species of origin, especially human, as well as functionally equivalent variants, having additions, deletions, and substitutions of either nucleotides or amino acids which do not significantly alter the functional activity of the protein as a receptor characterized by the binding activity identified above. Studies on human SR-BI show that human SR-BI is expressed in tissues similarly to murine SR-BI and has in vitro binding activity similar to murine SR-BI.

II. Methods of Regulation of SR-BI Cholesterol Transport to Alter Steroidogenesis.

It has now been demonstrated that SR-BI and the related SR-B proteins play critical roles in HDL lipid metabolism and cholesterol transport. SR-BI appears to be responsible for cholesterol delivery to steroidogenic tissues and liver, and actually transfers cholesterol from HDL particles through the liver cells and into the bile canniculi, where it is passed out into the intestine. Data indicates that SR-BI is also expressed in the intestinal mucosa although the location and amount appears to be correlated with stages of development.

As discussed above, the SR-BI proteins and antibodies and their DNAs can be used in screening of drugs which modulate the activity and/or the expression of SR-BI. These compounds can then regulate the amount of cholesteryl ester that is processed by the liver and steroidogenic tissues, and used as a means to lower steroid levels. Steroids produced by the body include sterols, bile acids, certain hormones including reproductive hormones, such as estrogen, progesterone and testosterone, and adrenal hormones. The adrenal cortical hormones, the androgens, and the estrogens are the major lipid-soluble steroid hormones. Over 30 steroids are made by the adrenal cortex, including the glucocortiocoids, mineralocorticoids, and the steroids like corticosterone. Cortisol is the most important of the glucocorticoids, opposing some of the actions of insulin and promoting gluconeogenesis. Aldosterone is the major mineralocorticoid, assisting in the maintainance of the water and salt balance in the body.

Alteration of the amounts of these steroids have clear applications as contraceptives and in the treatment of disorders characterized by the overproduction of steroids. Many of these are known. Some, such as Cushings' disease, are caused by hypersecretion of glucocorticoids. Addison's disease is caused by a deficiency in the secretion of adrenal cortical hormones.

The data provided in Example 6, below, shows that deletion of SR-BI can be effective as a contraceptive, without apparent harmful effects.

Other disorders are treated by lowering of hormone levels. Common diseases include endometriosis, fibroid tumors, and cancers of the breast and other reproductive organs. Inhibitors of SR-BI transport or binding can be used to treat these patients, to thereby lower estrogen or testerone levels as necessary to treat the disorder.

Nucleotide Molecules

Preferred uses for the nucleotide sequences shown in the Sequence Listings below, are for the screening of drugs altering binding of ligand or selective uptake of lipid from a ligand by the scavenger receptor proteins, or expression or translation of the SR-BI protein.

The preferred size of a hybridization probe is from 10 nucleotides to 100,000 nucleotides in length. Below 10 nucleotides, hybridized systems are not stable and will begin to denature above 20° C. Above 100,000 nucleotides, one finds that hybridization (renaturation) becomes a much slower and incomplete process, as described in greater detail in the text MOLECULAR GENETICS, Stent, G. S. and R. Calender, pp. 213-219 (1971). Ideally, the probe should be from 20 to 10,000 nucleotides. Smaller nucleotide sequences (20-100) lend themselves to production by automated organic synthetic techniques. Sequences from 100-10,000 nucleotides can be obtained from appropriate restriction endonuclease treatments. The labeling of the smaller probes with the relatively bulky chemiluminescent moieties may in some cases interfere with the hybridization process.

Screening for Drugs Modifying or Altering the Extent of Receptor Function or Expression

The receptor proteins are useful as targets for compounds which turn on, or off, or otherwise regulate binding to these receptors. The assays described below clearly provide routine methodology by which a compound can be tested for an inhibitory effect on binding of a specific compound, such as a radiolabeled modified HDL and LDL or fluorescently labelled ligands. The in vitro studies of compounds which appear to inhibit binding to and/or selective uptake by the receptors are then confirmed by animal testing. Since the molecules are so highly evolutionarily conserved, it is possible to conduct studies in laboratory animals such as mice to predict the effects in humans.

Studies based on inhibition of binding are predictive for indirect effects of alteration of receptor binding.

The following assays can be used to screen for compounds which are effective in methods for alter SR-BI expression, concentration, or transport of cholesterol. Since cholesterol is the basis for the production of steroids in the steroidogenic tissues, inhibition of cholesterol transport, via SR-BI, is effective in achieving contraception or limiting production of steroids.

Assays for Alterations in SR-BI Binding or Expression

Northern blot analysis of murine tissues shows that SR-BI is most abundantly expressed in adrenal, ovary, liver, testes, and fat and is present at lower levels in some other tissues. SR-BI MRNA expression is induced upon differentiation of 3T3-L1 cells into adipocytes. Both SR-BI and CD36 display high affinity binding for acetylated LDL with an apparent dissociation constant in the range of approximately 5 μg protein/ml. The ligand binding specificities of CD36 and SR-BI, determined by competition assays, are similar, but not identical: both bind modified proteins (acetylated LDL, maleylated BSA), but not the broad array of other polyanions (e.g. fucoidin, polyinosinic acid, polyguanosinic acid) which are ligands of the class A receptors. SR-BI displays high affinity and saturable binding of HDL which is not accompanied by cellular degradation of the HDL. HDL inhibits binding of AcLDL to CD36, suggesting that it binds HDL, similarly to SR-BI. Native LDL, which does not compete for the binding of acetylated LDL to either class A receptors or CD36, competes well for binding of LDL to SR-BI but is a very poor competitor of HDL binding.

¹²⁵I-AcLDL Binding, Uptake and Degradation Assays.

Scavenger receptor activities at 37° C. for some ligands can be measured by ligand binding, uptake and degradation assays as described by Krieger, Cell 33, 413-422, 1983; and Freeman et al., 1991). The values for binding and uptake are combined and are presented as binding plus uptake observed after a 5 hour incubation and are expressed as ng of ¹²⁵I-AcLDL protein per 5 hr per mg cell protein. Degradation activity is expressed as ng of ¹²⁵I-AcLDL protein degraded in 5 hours per mg of cell protein. The specific, high affinity values represent the differences between the results obtained in the presence (single determinations) and absence (duplicate determinations) of excess unlabeled competing ligand. Cell surface 4° C. binding is assayed using either method A or method B as indicated. In method A, cells are prechilled on ice for 15 min, re-fed with ¹²⁵I-AcLDL in ice-cold medium B supplemented with 10% (v/v) fetal bovine serum, with or without 75-200 μg/ml unlabeled M-BSA, and incubated 2 hr at 4° C. on a shaker. Cells are then washed rapidly three times with Tris wash buffer (50 mM Tris-HCl, 0.15 M NaCl, pH 7.4) containing 2 mg/ml BSA, followed by two 5 min washes, and two rapid washes with Tris wash buffer without BSA. The cells are solubilized in 1 ml of 0.1 N NaOH for 20 min at room temperature on a shaker, 30 μl are removed for protein determination, and the radioactivity in the remainder is determined using a LKB gamma counter. Method B differs from method A in that the cells are prechilled for 45 minutes, the medium contains 10 mM HEPES and 5% (v/v) human lipoprotein-deficient serum rather than fetal bovine serum, and the cell-associated radioactivity released by treatment with dextran sulfate is measured as described by Krieger, 1983; Freeman et al., 1991).

Northern Blot Analysis.

0.5 micrograms of poly(A)+ RNA prepared from different murine tissues or from 3T3-L1 cells on zero, two, four, six or eight days after initiation of differentiation into adipocytes as described by Baldini et al., 1992 Proc. Natl. Acad. Sci. U.S.A. 89, 5049-5052, is fractionated on a formaldehyde/agarose gel (1.0%) and then blotted and fixed onto a Biotrans™ nylon membrane. The blots are hybridized with probes that are ³²P-labeled (2×10⁶ dpm/ml, random-primed labeling system). The hybridization and washing conditions, at 42° C. and 50° C., respectively, are performed as described by Charron et al., 1989 Proc. Natl. Acad. Sci. U.S.A. 86, 2535-2539. The probe for SR-BI MRNA analysis was a 0.6 kb BamHI fragment from the cDNAs coding region. The coding region of murine cytosolic hsp70 gene (Hunt and Calderwood, 1990 Gene 87, 199-204) is used as a control probe for equal mRNA loading.

SR-BI protein in tissues is detected by blotting with polyclonal antibodies to SR-BI.

HDL Binding Studies

HDL and VLDL binding to SR-BI and CD36 are conducted as described for LDL and modified LDL.

Studies conducted to determine if the HDL which is bound to SR-BI is degraded or recycled and if lipid which is bound to the HDL is transferred into the cells are conducted using fluorescent lipid-labeled HDL, ³H-cholesteryl ester labeled HDL and ¹²⁵I-HDL added to cultures of transfected or untransfected cells at a single concentration (10 μg protein/ml). HDL associated with the cells is measured over time. A steady state is reached in approximately thirty minutes to one hour. A fluorescent ligand, DiI, or ³H-cholesterol ester is used as a marker for lipid (for example, cholesterol or cholesterol ester) uptake by the cell. Increasing concentration of DiI indicates that lipid is being transferred from the HDL to the receptor, then being internalized by the cell. The DiI-depleted HDL is then released and replaced by another HDL molecule.

HDL Binding to SR-BI

Competition binding studies demonstrate that HDL and VLDL (400 μg/ml) competitively inhibit binding of ¹²⁵I-AcLDL to SR-BI. Direct binding of ¹²⁵I-HDL to cells expressing SR-BI is also determined.

Tissue Distribution of SR-BI

To explore the physiological functions of SR-BI, the tissue distribution of SR-BI was determined in murine tissues, both in control animals and estrogen treated animals, as described in the following examples. Each lane is loaded with 0.5 μg of poly(A)+ RNA prepared from various murine tissues: kidney, liver, adrenals, ovaries, brain, testis, fat, diaphragm, heart, lung, spleen, or other tissue. The blots are hybridized with a 750 base pair fragment of the coding region of SR-BI. SR-BI MRNA is most highly expressed in adrenals, ovary and liver is moderately or highly expressed in fat depended on the source and is expressed at lower levels in other tissues. Blots using polyclonal antibodies to a cytoplasmic region of SR-BI demonstrate that very high levels of protein are present in liver, adrenal tissues, and ovary in mice and rats, but only very low or undetectable levels are present in either white or brown fat, muscle or a variety of other tissues. Bands in the rat tissues were present at approximately 82 kD. In the mouse tissues, the 82 kD form observed in the liver and steroidogenic tissues is the same size observed in SR-BI-transfected cultured cells.

Assays for testing compounds for useful activity can be based solely on interaction with the receptor protein, preferably expressed on the surface of transfected cells such as those described above, although proteins in solution or immobilized on inert substrates can also be utilized, where the indication is inhibition or increase in binding of lipoproteins. For example, these assays can be used to screen for compounds which selectively alter SR-BI levels in different tissue, or which alter SR-BI binding in vitro.

Alternatively, the assays can be based on interaction with the gene sequence encoding the receptor protein, preferably the regulatory sequences directing expression of the receptor protein. For example, antisense which binds to the regulatory sequences, and/or to the protein encoding sequences can be synthesized using standard oligonucleotide synthetic chemistry. The antisense can be stabilized for pharmaceutical use using standard methodology (encapsulation in a liposome or microsphere; introduction of modified nucleotides that are resistant to degradation or groups which increase resistance to endonucleases, such as phosphorothiodates and methylation), then screened initially for alteration of receptor activity in transfected or naturally occurring cells which express the receptor, then in vivo in laboratory animals. Typically, the antisense would inhibit expression. However, sequences which block those sequences which “turn off” synthesis can also be targeted.

The receptor protein for study can be isolated from either naturally occurring cells or cells which have been genetically engineered to express the receptor, as described in the examples above. In the preferred embodiment, the cells would have been engineered using the intact gene.

Random generation of receptor or receptor encoding sequence binding molecules.

Molecules with a given function, catalytic or ligand-binding, can be selected for from a complex mixture of random molecules in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. For example, by repeated cycles of affinity chromatography and PCR amplification of the molecules bound to the ligand on the column, Ellington and Szostak (1990) estimated that 1 in 10¹⁰ RNA molecules folded in such a way as to bind a given ligand. DNA molecules with such ligand-binding behavior have been isolated (Ellington and Szostak, 1992; Bock et al, 1992).

Computer Assisted Drug Design

Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modelling systems are the CHARMm and QUANTA programs, Polygen Corporation, Waltham, MA. CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model receptor for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of drugs specific to regions of DNA or RNA, once that region is identified.

Although described above with reference to design and generation of compounds which could alter binding and therefore cholesterol transport, one could also screen libraries of known compounds, including natural products or synthetic chemicals, and biologically active materials, including proteins, for compounds which are inhibitors or activators.

Generation of Nucleic Acid Regulators

Nucleic acid molecules containing the 5′ regulatory sequences of the receptor genes can be used to regulate or inhibit gene expression in vivo. Vectors, including both plasmid and eukaryotic viral vectors, may be used to express a particular recombinant 5′ flanking region-gene construct in cells depending on the preference and judgment of the skilled practitioner (see, e.g., Sambrook et al., Chapter 16). Furthermore, a number of viral and nonviral vectors are being developed that enable the introduction of nucleic acid sequences in vivo (see, e.g., Mulligan, 1993 Science, 260, 926-932; U.S. Pat. No. 4,980,286; U.S. Pat. No. 4,868,116; incorporated herein by reference). For example, a delivery system in which nucleic acid is encapsulated in cationic liposomes which can be injected intravenously into a mammal has been used to introduce DNA into the cells of multiple tissues of adult mice, including endothelium and bone marrow (see, e.g., Zhu et al., 1993 Science 261, 209-211; incorporated herein by reference).

The 5′ flanking sequences of the receptor gene can also be used to inhibit the expression of the receptor. For example, an antisense RNA of all or a portion of the 5′ flanking region of the receptor gene can be used to inhibit expression of the receptor in vivo. Expression vectors (e.g., retroviral or adenoviral expression vectors) are already in the art which can be used to generate an antisense RNA of a selected DNA sequence which is expressed in a cell (see, e.g., U.S. Pat. No. 4,868,116; U.S. Pat. No. 4,980,286). Accordingly, DNA containing all or a portion of the sequence of the 5′ flanking region of the receptor gene can be inserted into an appropriate expression vector so that upon passage into the cell, the transcription of the inserted DNA yields an antisense RNA that is complementary to the MRNA transcript of the receptor protein gene normally found in the cell. This antisense RNA transcript of the inserted DNA can then base-pair with the normal MRNA transcript found in the cell and thereby prevent the mRNA from being translated. It is of course necessary to select sequences of the 5′ flanking region that are downstream from the transcriptional start sites for the receptor protein gene to ensure that the antisense RNA contains complementary sequences present on the mRNA.

Antisense RNA can be generated in vitro also, and then inserted into cells. Oligonucleotides can be synthesized on an automated synthesizer (e.g., Model 8700 automated synthesizer of Milligen-Biosearch, Burlington, Mass. or ABI Model 380B). In addition, antisense deoxyoligonucleotides have been shown to be effective in inhibiting gene transcription and viral replication (see e.g., Zamecnik et al., 1978 Proc. Natl. Acad. Sci. USA 75, 280-284; Zamecnik et al., 1986 Proc. Natl. Acad. Sci., 83, 4143-4146; Wickstrom et al., 1988 Proc. Natl. Acad. Sci. USA 85, 1028-1032; Crooke, 1993 FASEB J. 7, 533-539. Furthermore, recent work has shown that improved inhibition of expression of a gene by antisense oligonucleotides is possible if the antisense oligonucleotides contain modified nucleotides (see, e.g., Offensperger et. al., 1993 EMBO J. 12, 1257-1262 (in vivo inhibition of duck hepatitis B viral replication and gene expression by antisense phosphorothioate oligodeoxynucleotides); Rosenberg et al., PCT WO 93/01286 (synthesis of sulfurthioate oligonucleotides); Agrawal et al., 1988 Proc. Natl. Acad. Sci. USA 85, 7079-7083 (synthesis of antisense oligonucleoside phosphoramidates and phosphorothioates to inhibit replication of human immunodeficiency virus-1); Sarin et al., 1989 Proc. Natl. Acad. Sci. USA 85, 7448-7794 (synthesis of antisense methylphosphonate oligonucleotides); Shaw et al., 1991 Nucleic Acids Res 19, 747-750 (synthesis of 3′ exonuclease-resistant oligonucleotides containing 3′ terminal phosphoroamidate modifications); incorporated herein by reference).

The sequences of the 5′ flanking region of receptor protein gene can also be used in triple helix (triplex) gene therapy. Oligonucleotides complementary to gene promoter sequences on one of the strands of the DNA have been shown to bind promoter and regulatory sequences to form local triple nucleic acid helices which block transcription of the gene (see, e.g., 1989 Maher et al., Science 245, 725-730; Orson et al., 1991 Nucl. Acids Res. 19, 3435-3441; Postal et al., 1991 Proc. Natl. Acad. Sci. USA 88, 8227-8231; Cooney et al., 1988 Science 241, 456-459; Young et al., 1991 Proc. Natl. Acad. Sci. USA 88, 10023-10026; Duval-Valentin et al., 1992 Proc. Natl. Acad. Sci. USA 89, 504-508; 1992 Blume et al., Nucl. Acids Res. 20, 1777-1784; 1992 Grigoriev et al., J. Biol. Chem. 267, 3389-3395.

Both theoretical calculations and empirical findings have been reported which provide guidance for the design of oligonucleotides for use in oligonucleotide-directed triple helix formation to inhibit gene expression. For example, oligonucleotides should generally be greater than 14 nucleotides in length to ensure target sequence specificity (see, e.g., Maher et al., (1989); Grigoriev et al., (1992)). Also, many cells avidly take up oligonucleotides that are less than 50 nucleotides in length (see e.g., Orson et al., (1991); Holt et al., 1988 Mol. Cell. Biol. 8, 963-973; Wickstrom et al., 1988 Proc. Natl. Acad. Sci. USA 85, 1028-1032). To reduce susceptibility to intracellular degradation, for example by 3′ exonucleases, a free amine can be introduced to a 3′ terminal hydroxyl group of oligonucleotides without loss of sequence binding specificity (Orson et al., 1991). Furthermore, more stable triplexes are formed if any cytosines that may be present in the oligonucleotide are methylated, and also if an intercalating agent, such as an acridine derivative, is covalently attached to a 5′ terminal phosphate (e.g., via a pentamethylene bridge); again without loss of sequence specificity (Maher et al., (1989); Grigoriev et al., (1992).

Methods to produce or synthesize oligonucleotides are well known in the art. Such methods can range from standard enzymatic digestion followed by nucleotide fragment isolation (see e.g., Sambrook et al., Chapters 5, 6) to purely synthetic methods, for example, by the cyanoethyl phosphoramidite method using a Milligen or Beckman System lPlus DNA synthesizer (see also, Ikuta et al., in Ann. Rev. Biochem. 1984 53, 323-356 (phosphotriester and phosphite-triester methods); Narang et al., in Methods Enzymol., 65, 610-620 (1980) (phosphotriester method). Accordingly, DNA sequences of the 5′ flanking region of the receptor protein gene described herein can be used to design and construct oligonucleotides including a DNA sequence consisting essentially of at least 15 consecutive nucleotides, with or without base modifications or intercalating agent derivatives, for use in forming triple helices specifically within the 5′ flanking region of a receptor protein gene in order to inhibit expression of the gene.

In some cases it may be advantageous to insert enhancers or multiple copies of the regulatory sequences into an expression system to facilitate screening of methods and reagents for manipulation of expression.

Preparation of Receptor Protein Fragments

Compounds which are effective for blocking binding of the receptor to the cholesterol-HDL can also consist of fragments of the receptor proteins including the extracellular region of the receptor which binds to the lipoprotein, expressed recombinantly and cleaved by enzymatic digest or expressed from a sequence encoding a peptide of less than the full length receptor protein. These will typically be soluble proteins, i.e., not including the transmembrane and cytoplasmic regions, although smaller portions determined in the assays described above to inhibit or compete for binding to the receptor proteins can also be utilized. It is a routine matter to make appropriate receptor protein fragments, test for binding, and then utilize. The preferred fragments are of human origin, in order to minimize potential immunological response. The peptides can be as short as five to eight amino acids in length and are easily prepared by standard techniques. They can also be modified to increase in vivo half-life, by chemical modification of the amino acids or by attachment to a carrier molecule or inert substrate. Based on studies with other peptide fragments blocking receptor binding, the IC₅₀, the dose of peptide required to inhibit binding by 50%, ranges from about 50 μM to about 300 μM, depending on the peptides. These ranges are well within the effective concentrations for the in vivo administration of peptides, based on comparison with the RGD-containing peptides, described, for example, in U.S. Pat. No. 4,792,525 to Ruoslaghti, et al., used in vivo to alter cell attachment and phagocytosis.

The peptides can also be conjugated to a carrier protein such as keyhole limpet hemocyanin by its N-terminal cysteine by standard procedures such as the commercial Imject kit from Pierce Chemicals or expressed as a fusion protein, which may have increased efficacy. As noted above, the peptides can be prepared by proteolytic cleavage of the receptor proteins, or, preferably, by synthetic means. These methods are known to those skilled in the art. An example is the solid phase synthesis described by J. Merrifield, 1964 J. Am. Chem. Soc. 85, 2149, used in U.S. Pat. No. 4,792,525, and described in U.S. Pat. No. 4,244,946, wherein a protected alpha-amino acid is coupled to a suitable resin, to initiate synthesis of a peptide starting from the C-terminus of the peptide. Other methods of synthesis are described in U.S. Pat. No. 4,305,872 and 4,316,891. These methods can be used to synthesize peptides having identical sequence to the receptor proteins described herein, or substitutions or additions of amino acids, which can be screened for activity as described above.

The peptide can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Peptides containing cyclopropyl amino acids, or amino acids derivatized in a similar fashion, can also be used. These peptides retain their original activity but have increased half-lives in vivo. Methods known for modifying amino acids, and their use, are known to those skilled in the art, for example, as described in U.S. Pat. No. 4,629,784 to Stammer.

The peptides are generally active when administered parenterally in amounts above about 1 μg/kg of body weight. Based on extrapolation from other proteins for treatment of most inflammatory disorders, the dosage range will be between 0.1 to 70 mg/kg of body weight. This dosage will be dependent, in part, on whether one or more peptides are administered.

Pharmaceutical Compositions

Compounds which alter receptor protein binding are preferably administered in a pharmaceutically acceptable vehicle. Suitable pharmaceutical vehicles are known to those skilled in the art. For parenteral administration, the compound will usually be dissolved or suspended in sterile water or saline. For enteral administration, the compound will be incorporated into an inert carrier in tablet, liquid, or capsular form. Suitable carriers may be starches or sugars and include lubricants, flavorings, binders, and other materials of the same nature. The compounds can also be administered locally by topical application of a solution, cream, gel, or polymeric material (for example, a Pluronic™, BASF).

Alternatively, the compound may be administered in liposomes or microspheres (or microparticles). Methods for preparing liposomes and microspheres for administration to a patient are known to those skilled in the art. U.S. Pat. No. 4,789,734 describe methods for encapsulating biological materials in liposomes. Essentially, the material is dissolved in an aqueous solution, the appropriate phospholipids and lipids added, along with surfactants if required, and the material dialyzed or sonicated, as necessary.

A review of known methods is by G. Gregoriadis, Chapter 14. “Liposomes”, Drug Carriers in Biology and Medicine pp. 287-341 (Academic Press, 1979). Microspheres formed of polymers or proteins are well known to those skilled in the art, and can be tailored for passage through the gastrointestinal tract directly into the bloodstream. Alternatively, the compound can be incorporated and the microspheres, or composite of microspheres, implanted for slow release over a period of time, ranging from days to months. See, for example, U.S. Pat. Nos. 4,906,474, 4,925,673, and 3,625,214.

The pharmaceutical compositions are administered in an amount effective to modify the steroidal levels. These are readily determined by measuring blood, urine and/or tissue samples using clinically available tests. The exact dosages can be determined based on the use of animal models which are accepted as predictive of the effects of drugs on steroid levels, for example, of contraceptives or cortisone.

Generation of Transgenic Animals for Screening

With the knowledge of the cDNA encoding SR-BI and regulatory sequences regulating expression thereof, it is possible to generate transgenic animals, especially rodents, for testing the compounds which can alter SR-BI expression, translation or function in a desired manner. This procedure for transient overexpression in animals following infection with adenoviral vectors is described below in the examples.

There are basically two types of animals which are useful: those not expressing functional SR-BI, which are useful for testing of drugs which may work better in combination with an inhibitor of SR-BI to control levels of lipid, cholesterol, lipoprotein or components thereof, and those which overexpress SR-BI, either in those tissues which already express the protein or in those tissues where only low levels are naturally expressed.

The animals in the first group are preferably made using techniques that result in “knocking out” of the gene for SR-BI, although in the preferred case this will be incomplete, either only in certain tissues, or only to a reduced amount. These animals are preferably made using a construct that includes complementary nucleotide sequence to the SR-BI gene, but does not encode functional SR-BI, and is most preferably used with embryonic stem cells to create chimeras. Animals which are heterozygous for the defective gene can also be obtained by breeding a homozygote normal with an animal which is defective in production of SR-BI.

The animals in the second group are preferably made using a construct that includes a tissue specific promoter, of which many are available and described in the literature, or an unregulated promoter or one which is modified to increase expression as compared with the native promoter. The regulatory sequences for the SR-BI gene can be obtained using standard techniques based on screening of an appropriate library with the CDNA encoding SR-BI. These animals are most preferably made using standard microinjection techniques.

These manipulations are performed by insertion of cDNA or genomic DNA into the embryo using microinjection or other techniques known to those skilled in the art such as electroporation, as described below. The DNA is selected on the basis of the purpose for which it is intended: to inactivate the gene encoding an SR-BI or to overexpress or express in a different tissue the gene encoding SR-BI. The SR-BI encoding gene can be modified by homologous recombination with a DNA for a defective SR-BI, such as one containing within the coding sequence an antibiotic marker, which can then be used for selection purposes.

Animal Sources

Animals suitable for transgenic experiments can be obtained from standard commercial sources. These include animals such as mice and rats for testing of genetic manipulation procedures, as well as larger animals such as pigs, cows, sheep, goats, and other animals that have been genetically engineered using techniques known to those skilled in the art. These techniques are briefly summarized below based principally on manipulation of mice and rats.

Microinjection Procedures

The procedures for manipulation of the embryo and for microinjection of DNA are described in detail in Hogan et al. Manipulating the mouse embryo, Cold Spring Harbor Laboratory, Cold Spring Harbor, N.Y. (1986), the teachings of which are incorporated herein. These techniques are readily applicable to embryos of other animal species, and, although the success rate is lower, it is considered to be a routine practice to those skilled in this art.

Transgenic Animals

Female animals are induced to superovulate using methodology adapted from the standard techniques used with mice, that is, with an injection of pregnant mare serum gonadotrophin (PMSG; Sigma) followed 48 hours later by an injection of human chorionic gonadotrophin (hCG; Sigma). Females are placed with males immediately after hCG injection. Approximately one day after hCG, the mated females are sacrificed and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection.

Randomly cycling adult females are mated with vasectomized males to induce a false pregnancy, at the same time as donor females. At the time of embryo transfer, the recipient females are anesthetized and the oviducts are exposed by an incision through the body wall directly over the oviduct. The ovarian bursa is opened and the embryos to be transferred are inserted into the infundibulum. After the transfer, the incision is closed by suturing.

Embryonic Stem (ES) Cell Methods Introduction of cDNA into ES Cells:

Methods for the culturing of ES cells and the subsequent production of transgenic animals, the introduction of DNA into ES cells by a variety of methods such as electroporation, calcium phosphate/DNA precipitation, and direct injection are described in detail in Teratocarcinomas and embryonic stem cells, a practical approach, ed. E. J. Robertson, (IRL Press 1987), the teachings of which are incorporated herein. Selection of the desired clone of transgene-containing ES cells is accomplished through one of several means. In cases involving sequence specific gene integration, a nucleic acid sequence for recombination with the SR-BI gene or sequences for controlling expression thereof is co-precipitated with a gene encoding a marker such as neomycin resistance. Transfection is carried out by one of several methods described in detail in Lovell-Badge, in Teratocarcinomas and embrvonic stem cells, a practical approach, ed. E. J. Robertson, (IRL Press 1987) or in Potter et al Proc. Natl. Acad. Sci. USA 81, 7161 (1984). Calcium phosphate/DNA precipitation, direct injection, and electroporation are the preferred methods. In these procedures, a number of ES cells, for example, 0.5×10⁶, are plated into tissue culture dishes and transfected with a mixture of the linearized nucleic acid sequence and 1 mg of pSV2neo DNA (Southern and Berg, J. Mol. Appl. Gen. 1:327-341 (1982)) precipitated in the presence of 50 mg lipofectin in a final volume of 100 μl. The cells are fed with selection medium containing 10% fetal bovine serum in DMEM supplemented with an antibiotic such as G418 (between 200 and 500 μg/ml). Colonies of cells resistant to G418 are isolated using cloning rings and expanded. DNA is extracted from drug resistant clones and Southern blotting experiments using the nucleic acid sequence as a probe are used to identify those clones carrying the desired nucleic acid sequences. In some experiments, PCR methods are used to identify the clones of interest.

DNA molecules introduced into ES cells can also be integrated into the chromosome through the process of homologous recombination, described by Capecchi, (1989). Direct injection results in a high efficiency of integration. Desired clones are identified through PCR of DNA prepared from pools of injected ES cells. Positive cells within the pools are identified by PCR subsequent to cell cloning (Zimmer and Gruss, Nature 338, 150-153 (1989)). DNA introduction by electroporation is less efficient and requires a selection step. Methods for positive selection of the recombination event (i.e., neo resistance) and dual positive-negative selection (i.e., neo resistance and ganciclovir resistance) and the subsequent identification of the desired clones by PCR have been described by Joyner et al., Nature 338, 153-156 (1989) and Capecchi, (1989), the teachings of which are incorporated herein.

Embryo Recovery and ES Cell Injection

Naturally cycling or superovulated females mated with males are used to harvest embryos for the injection of ES cells. Embryos of the appropriate age are recovered after successful mating. Embryos are flushed from the uterine horns of mated females and placed in Dulbecco's modified essential medium plus 10% calf serum for injection with ES cells. Approximately 10-20 ES cells are injected into blastocysts using a glass microneedle with an internal diameter of approximately 20 μm.

Transfer of Embryos to Pseudopregnant Females

Randomly cycling adult females are paired with vasectomized males. Recipient females are mated such that they will be at 2.5 to 3.5 days post-mating (for mice, or later for larger animals) when required for implantation with blastocysts containing ES cells. At the time of embryo transfer, the recipient females are anesthetized. The ovaries are exposed by making an incision in the body wall directly over the oviduct and the ovary and uterus are externalized. A hole is made in the uterine horn with a needle through which the blastocysts are transferred. After the transfer, the ovary and uterus are pushed back into the body and the incision is closed by suturing. This procedure is repeated on the opposite side if additional transfers are to be made.

Identification of Transgenic Animals.

Samples (1-2 cm of mouse tails) are removed from young animals. For larger animals, blood or other tissue can be used. To test for chimeras in the homologous recombination experiments, i.e., to look for contribution of the targeted ES cells to the animals, coat color has been used in mice, although blood could be examined in larger animals. DNA is prepared and analyzed by both Southern blot and PCR to detect transgenic founder (F₀) animals and their progeny (F₁ and F₂).

Once the transgenic animals are identified, lines are established by conventional breeding and used as the donors for tissue removal and implantation using standard techniques for implantation into humans.

The present invention will be further understood by reference to the following non-limiting examples.

### EXAMPLE 1

### Uptake of HDL lipid mediated by SR-BI

The fates of the lipid and apoprotein components of HDL after interaction with mSR-BI were compared by examining the time-course of cell association of labeled HDL, where either the proteins (¹²⁵I) or the lipids ([³H]cholesteryl oleate or DiI (a fluorescent lipid)) were labeled.

A. Uptake of Labeled HDL by SR-BI

Methods

On day 0, IdlA cells and ldlA[mSR-BI] cells were plated in 6-well dishes (250,000 cells/well) in Ham's F-12 medium containing 100 units/ml penicillin, 100 μg/ml streptomycin, and 2 mM glutamine (medium A) supplemented with 5% fetal bovine serum (A-FBS) without or with 0.25 mg/ml G418, respectively. Assays were performed on day 2.

HDL and LDL were prepared from human plasma by zonal centrifugation (Chung, et al. in Methods of Enzymology, Ed J. P. Segrest and J. J. Albers (Academic Press, Inc. Orlando, Fla. 1986) Vol. 128, pp. 181-209. SDS-PAGE showed that the only major proteins in the HDL were apoaI and apo AII (the mass ratio of Ai:AII was at least 3:1). Apo E was either undetectable or present in trace amounts. For some experiments the apo E was removed using a HiTrap Heparin column (Pharmacia) essentially as described in ‘Lipoprotein Analysis: A Practical Approach’., Ed. C. A. Converse and E. R. Skinner (Oxford University Press, 1992). The mass ratio of cholesterol:protein in HDL was assumed to be 1:4. HDL was iodinated by the iodobead method (Pierce) as follows: 2 mg of HDL in 0.2 ml phosphate buffered saline (Ca²⁺, Mg²⁺ free) was added to 0.25 ml of 0.3 M sodium phosphate buffer, pH 7.4 containing 2 iodobeads and 1 mCi ¹²⁵I-NaI. After 5 min at room temperature, the reaction was quenched with 25 μl saturated L-tyrosine (in water) and dialyzed extensively against 0.15 M NaCl, 0.3 mM EDTA, pH 7.4. The specific activities ranged from 60 to 360 cpm/ng protein. [³H]cholesteryl ester labeled HDL was a gift from Alan Tall (Columbia University, Jammett and Tall, J. Biol. Chem. 260, 6687,(1985)).

DiI(D-282, 1,1′-dioctadecyl-3,3,3′,3′-tetramethylindocarbocyanine perchlorate) was from Molecular Probes (Eugene, OR). DiI -HDL was prepared essentially as described previously for Dil-LDL by Pitas, et al., Arterioclerosis 1, 177 (1981)). The protein content of lipoproteins and cells was determined by the method of Lowry J. Biol. Chem. 193, 265 (1951)).

To determine the concentration dependence of ¹²⁵I-HDL cell association (ng ¹²⁵I-HDL protein associated/1.5 hr/mg cell protein), cells were refed with ¹²⁵I-HDL (250 cpm/ng protein)) in medium A containing 0.5% (w/v) fatty acid free bovine serum albumin (FAF-BSA) (medium B) with or without unlabeled HDL (40-fold excess), and incubated for 1.5 hr at 37° C. in a 5% CO₂ humidified incubator. Cells were then chilled, rapidly washed twice with 2 ml of ice cold Tris wash buffer (50 mM Tris-HCl, 0.15 M NaCl, pH 7.4) containing 2 mg/ml BSA, once with Tris wash buffer without BSA, and radioactivity and protein determinations were made. The specific values were calculated based on the differences between the results obtained in the presence (single determinations, nonspecific activity) and absence (duplicate determinations, total activity) of excess unlabeled HDL. The time course of cell association of ¹²⁵I-HDL. Cells were incubated with 20 μg protein/ml of ¹²⁵I-HDL (220 cpm/ng protein) at 37° C. was determined and specific cell association (ng draft ¹²⁵I-HDL protein associated/mg cell protein) was determined as described above. The time course of ¹²⁵I-HDL degradation was then measured. Cells were incubated with 10 μg protein/ml of ¹²⁵I-HDL (64 cpm/ng protein) and specific cellular degradation (ng of 125I-HDL protein degraded per mg of cell protein) to acid soluble products was determined.

To determine the kinetics of selective uptake of HDL lipid by mSR-BI, untransfected and SR-BI expressing cells were plated on day 0 and on day 2 they were incubated at 37° C. with ¹²⁵I-HDL (10 μg of protein/ml, 64 cpm/ng protein), [³H]-cholesteryl oleate-labeled HDL (approximately 8.8 μg of protein/ml, 15 cpm/ng cholesteryl ester), or DiI-labeled HDL (10 μg of protein/ml), and cell associated label was quantified. [³H]-cholesteryl oleate was extracted with isopropyl alcohol for 30 minutes at room temperature, and radioactivity was measured in Scintiverse II (Fisher) scintillation mixture. DiI was extracted by *disolving the cells in DMSO and measuring the fluorescence in a Hitachi model F-4500 fluorescence spectrophotometer at 550 nm excitation, 565 nm emission and comparing to standards prepared of DiI-HDL dissolved in DMSO.

To determine if the SR-BI-mediated transfer [³H]cholesteryl ester from labeled HDL represented net transfer of this lipid rather than exchange, the cholesterol contents of the cells after incubation with or without unlabeled HDL (20 μg protein/ml, 5 hours) was compared. On day 2 after plating, cells were incubated for 5 hours at 37° C. in medium B in the presence or absence of unlabeled HDL (20 μg protein/ml), washed as described above, and lipids were extracted twice with hexane/isopropanol (3:2, 3 ml, 30 minutes). Extracts were pooled, backextracted with 1 ml water, and dried by rotary evaporation. Total (free and esterified) cholesterol masses (averages of six replicates) were determined using an enzymatic assay (Sigma Diagnostics, St. Louis, Mo.). The protein contents of the sample were estimated by analysis of replicate cultures. The values of total cholesterol (μg/mg cell protein+SEM) for the cells incubated without HDL were 20.5 +0.3 (ldlA) and 23.0+0.4 (ldlA[mSR-BI]).

Results

²⁵I-HDL specifically associated with SR-BI expressing cells with high affinity (kD approximately 30 μg of protein/ml) and saturability. Control cells exhibited substantially less ¹²⁵I-HDL association. Association was very rapid, reaching a steady state in less than 1 hour. Despite this high affinity and saturable binding, the ¹²⁵I-labeled protein components of HDL were not degraded after interaction with SR-BI expressing cells.

The kinetics of association of the protein components of HDL differed greatly from those of the lipids. Only a small fraction (less than 0.5%) of the total label in the ¹²⁵I-HDL was bound to the transfected cells in a 5 hour period. Cell-associated ¹²⁵I-HDL reached a steady-state (approximately 200 ng protein/mg cell protein at 10 μg HDL protein/ml) in less than one hour. In contrast, cell association of the lipid-labeled component of HDL ([³H]cholesteryl oleate or DiI) continuously increased throughout the incubation. The kinetics of [³H]cholesterol ester and DiI transfer to the cells were similar. Approximately 18% of the total labeled lipids in HDL added to the incubation media were specifically associated with transfected cells at the end of the 5 hour incubations. Untransfected cells displayed little lipid or protein association. Thus, there was selective transfer of the lipid, but not the protein, components of HDL to the cells expressing mSR-BI.

To determine if the transfer of [³H]cholesteryl ester from labeled HDL represented net transfer of this lipid rather than exchange, the cholesterol contents of the cells after incubation with or without unlabeled HDL (20 μg protein/ml, 5 hrs) was compared. In the transfected cells, incubation with HDL resulted in a 20% increase (4.6 μg cholesterol/mg of cell protein) in total cellular cholesterol (free and esterified). This increase corresponded to a transfer of approximately 21% of the HDL-cholesterol added to the incubation medium and was comparable to the amounts of labeled lipid transferred from either [³H]cholesteryl oleate-HDL or DiI-HDL. In contrast, there was no statistically significant HDL-dependent increase in the cholesterol content of the untransfected cells (less than 0.2 μg cholesterol/mg of cell protein). These results suggest that 1) mSR-BI mediated net mass transfer of HDL cholesteryl ester, 2) this transfer was quantitatively similar to that previously reported for a murine adrenal cell line (Y1-BS1), and 3) under these conditions, the fluorescent or radiolabeled lipids in HDL can serve as reasonable reporters for total cholesterol transfer.

B. Uptake of Fluorescent-labeled Lipid

To begin to examine the cellular pathway of selective lipid delivery mediated by mSR-BI, the initial distribution of fluorescent lipid (DiI) delivered via the classic LDL receptor pathway with that of the mSR-BI pathway was compared.

Methods

On day 0, LDL receptor-positive wild-type CHO, mSR-BI transfected ldlA[mSR-BI], and receptor-negative ldlA cells were plated in medium A containing 5% FBS on coverslips coated with poly-D-lysine (MW greater than 300,000, Sigma) as per the manufacturers instructions. A 600 bp probe from the hamster SR-BI cDNA described by Acton, et al., J. Biol. Chem. 269, 21003 (1994), the teachings of which are incorporated herein, was used to screen a murine 3T3-L1 adipocyte cDNA library. A clone containing the complete coding region was isolated and this region was sequenced on both strands; the sequence had 89% predicted amino acid identity and 96% similarity to the hamster sequence and 79% predicted amino acid identity and 91% similarity to the human sequence, CLA1 (Calvo and Vega, J. Biol. Chem. 268, 18929 (1993), the teachings of which are incorporated herein. The expression vector pmSR-BI-77 was generated from this clone and, using previously described methods, transfected into an LDL receptor-negative mutant CHO cell line, ldlA, to generate stable, receptor-positive transfectants. Flow cytometry after incubation with Dil-labeled acetylated LDL was used to isolate the subpopulation of cells, ldlA[mSR-BI] (colony 15), used here.

On day 1, the monolayers were refed with medium A containing 5% newborn calf lipoprotein-deficient serum. On day 3 the subconfluent cells were refed with the same medium containing either 10 μg protein/ml DiI-LDL (A) or 1 μg protein/ml of DiI-HDL (B and C) and incubated for 1 hr at 37° C. The coverslips were then washed once with phosphate buffered saline and the distribution of DiI was immediately recorded photographically using a Nikon fluorescence microscope with a rhodamine filter package.

Results

After LDL receptor-positive wild-type CHO cells were incubated with DiI-LDL (10 μg protein/ml) for one hr at 37° C., uptake via the classic LDL receptor resulted in a punctate pattern of labeling. This was typical for receptor-mediated endocytosis from coated pits and vesicles to endosomes and lysosomes. There was essentially no labeling by DiI-LDL of LDL receptornegative IdlA cells. DiI-HDL (1 μg protein/ml) labeling of ldlA[mSR-BI] cells was dramatically different rather than punctate fluorescence, there was diffuse staining over what appeared to be the entire surface of the transfected cells, with especially striking fluorescence at cell-cell interfaces. In addition, there was often a bright, apparently internal, concentration of fluorescence in a region adjacent to the nucleus. Even after 24 hrs of incubation, the Dil-fluorescence pattern in the mSR-BI transfectants did not resemble the punctate pattern seen for the LDL receptor pathway, although the pattern differed and possibly represents the subsequent redistribution of the dye away from the plasma membrane. Untransfected IdlA cells did not accumulate significant levels of dye from DiI-HDL. It is important to note that the initial distribution (less than or equal one hr) as well as the subsequent sites of accumulation of DiI, a positively charged lipid, may differ from those of cholesteryl ester, a neutral lipid. Indeed, it was observed that, after 48 hr of incubation with unlabeled HDL, neutral lipids transferred to the transfected cells apparently accumulated in small, well-defined cytoplasmic particles which stained with oil red O. Similarly, Reaven, et al., J. Lipid Res. 36, 1602 (1995), have reported the accumulation of a fluorescent cholesteryl ester derivative into cytoplasmic fat droplets in ovarian granuloma cells after a 9 hr incubation of the cells with labeled HDL. Taken together, these results indicate that the pathway by which mSR-BI mediates lipid transfer from HDL is distinct from the classic LDL receptor-mediated endocytic pathway and suggest that the HDL lipids may initially be transferred directly from the lipoprotein to the plasma membrane.

### EXAMPLE 2

### Tissue Distribution of SR-BI.

In vivo metabolic studies have established that the liver and steroidogenic tissues (adrenal and ovary) are the primary tissues involved in the selective uptake of HDL-cholesteryl esters, Glass, et al., Proc. Natl. Acad. Sci. USA 80, 5435 (1983), J. Biol. Chem. 260, 744 (1985), Khoo, et al., J. Lipid Res. 36, 593 (1995), Stein, et al., Biochim. Biophys. Acta 752, 98 (1983), Nestler, et al., Endocrinology 117, 502 (1985). Although numerous ligand blotting studies of these tissues have revealed a variety of HDL binding proteins ranging in size from 58 kD to 140 kD, none of these has directly been shown to mediate selective lipid uptake.

Methods

To determine the size of mSR-BI and its tissue distribution, a rabbit anti-mSR-BI polyclonal antibody was prepared by immunization of a 16 amino acid peptide (residues 495 to 509 from the predicted protein sequence of mSR-BI plus an additional N-terminal cysteine) coupled to keyhole limpet hemocyanin. This is referred to as anti-mSR-BI⁴⁹⁵ antiserum. The antiserum was used for immunoblot analysis of cultured cells and murine tissues.

Post-nuclear cell extracts from ldlA and ldlA[mSR-BI] cells and membranes (post-nuclear 100,000× g pellets) from murine tissues were isolated, reduced, and separated by 6.5% SDS-polyacrylamide gel electrophoresis (50 μg protein/lane), transferred to nitrocellulose and probed with a primary anti-mSR-BI⁴⁹⁵ antipeptide antibody (rabbit IgG fraction, 1:5000 dilution) and developed using a horseradish peroxidase labeled second antibody and ECL kit (5 min exposure, Amersham). Ponceau S staining was used as a control for gel loading and transfer.

Results

The antibody recognized an approximately 82 kD protein in transfected cells (ldlA[mSR-BI]) which was not present in the untransfected cells (ldlA). The predicted mass of the mSR-BI polypeptide is 57 kD, suggesting mSR-BI underwent significant co- and/or post-translational modification.

mSR-BI was most highly expressed in three tissues, liver and the steroidogenic ovary and adrenal glands. Significantly less mSR-BI protein was detected in testis, heart and mammary gland and essentially no expression was observed in other tissues, including brain, kidney, spleen, muscle, uterus, intestine, epididymal fat, lung and placenta. Thus, SR-BI is most abundantly expressed in precisely those tissues exhibiting selective cholesteryl ester transport in vivo.

A substantial signal in murine fat tissue and cultured adipocytes had been observed in previous Northern blotting studies using a hamster SR-BI cDNA probe. This lack of correlation with the immunoblot results reported here may be due to tissue specific differences in translational regulation or protein stability, or to cross hybridization of the hamster cDNA probe with mRNA of a related, but distinct, gene which is highly expressed in fat.

### EXAMPLE 3

### Analysis of Estrogen-Treated Rat Tissues for Expression of SR-BI

Methods

Tissues of estrogen-treated rats were screened for expression of SR-BI as described above following treatment of rats with 17-α-ethylenyl estradiol (estrogen). The rats were treated for five consecutive days with subcutaneous injections of 5 mg/kg 17-α-ethylenyl estradiol in propylene glycol or with propylene glycol alone (sham-injected).

Results

Immunoblots comparing the expression of SR-BI in rat tissues in estrogen-treated or sham-treated animals show the upregulation of SR-BI in rat adrenal membranes from animals treated with estrogen as compared with controls. There is no change in SR-BI levels in tissues showing trace signal, including lung as well as testes and skin. A longer exposure, comparing a SR-BI positive control and negative control, with liver tissues from estrogen treated and sham treated animals, and adrenal tissues from estrogen treated and sham treated animals show the same results.

Immunoblots comparing expression of the SR-BI and LDL receptor show that SR-BI expression was dramatically downregulated under conditions of tremendous upregulation of the LDL-receptor.

### EXAMPLE 4

### Analysis of Lipid Uptake in Estrogen Treated Animals

Methods

For analysis of HDL lipid uptake in vivo, rats were anesthetized with nembutal or metafane before injection of DiI-HDL (800 μg protein/kg) into the left jugular vein. 1 hour later the anesthetized animals were perfused with oxygenated HBSS. Frozen sections (12 μm thick) of sucrose-infiltrated tissues were prepared. Tissue sections were viewed and photographed with a Zeiss photomicroscope III with the appropriate filter package.

Results

When HDL labeled fluorescently with DI, as described above, was injected into the treated and control animals,animals receiving estrogen had significantly reduced levels of SR-BI expressed in the liver, and elevated levels of SR-BI and fluorescence in the ovaries. The uptake of lipid into adrenal tissues was also dramatically increased in the estrogen-treated animals.

### EXAMPLE 5

### Depletion of Blood Cholesterol Levels in Animals Transiently Overexpressing SR-BI

The in vivo effects of murine SR-BI (mSR-BI) on HDL and biliary cholesterol metabolism were studied in C57BL/6 mice that transiently overexpressed hepatic mSR-BI because of infection by intravenous infusion with a recombinant, replication defective adenovirus (Ad.mSR-BI). In the Ad.mSR-BI virus, the mSR-BI cDNA is under the control of the cytomegalovirus (CMV) immediate early enhancer/promotor. Controls included mice infected with a replication defective adenovirus lacking a cDNA transgene (Ad.ΔE1) exhibited modest levels of SR-BI expression, as determined by immunofluorescence microscopy and by immunoblotting. Three days post-infection, mSR-BI expression was dramatically increased in the livers of Ad.mSR-BI treated animals. Although the amount of mSR-BI protein decreased with time after infection, levels substantially above those of controls 21 days after infection were routinely observed. Much of the increase in mSR-BI expression appeared to be localized to the apical surfaces of the hepatocytes, with especially strong focal intensities suggesting high expression in the bile canaliculi. Sinusoidal staining was also observed.

The effects of hepatic SR-BI overexpression on plasma cholesterol levels are shown in Table 1. Infusion of control adenovirus had little or no effect on total cholesterol. In contrast, infusion of Ad.SR-BI resulted in dramatic decrease in plasma cholesterol by day 3, to approx. 14% of control levels. By day 7, cholesterol levels had increased to above preinfusion levels, and returned to baseline by day 21. Plasma levels of apoAI, the major protein component of HDL, mirrored total cholesterol levels in the initial decrease observed on day 3 (Table 1); in contrast, at later time points, apoAI levels increased but did not recover to pre-infusion levels until day 21.

TABLE 1
Plasma cholesterol and apoAI levels.

| | |
|---|---|
| Cholesterol (mg/dL) | apoAI (mg/dL) |

| Day | Ad.ΔE1 | Ad.SR-BI | Ad.ΔE1 | Ad.SR-BI |
|---|---|---|---|---|
| pre | 131.0 | 117.8 | 33.2 | 32.6 |
| 3 | 125.5 | 16.5 | 31.0 | 5.0 |
| 7 | 146.0 | 173.0 | 33.5 | 23.4 |
| 14 | 129.0 | 152.0 | 32.5 | 26.0 |
| 21 | 113.0 | 87.5 | 34.0 | 32.0 |

The numbers shown in the above table are averages for 2 to 8 mice/time point.

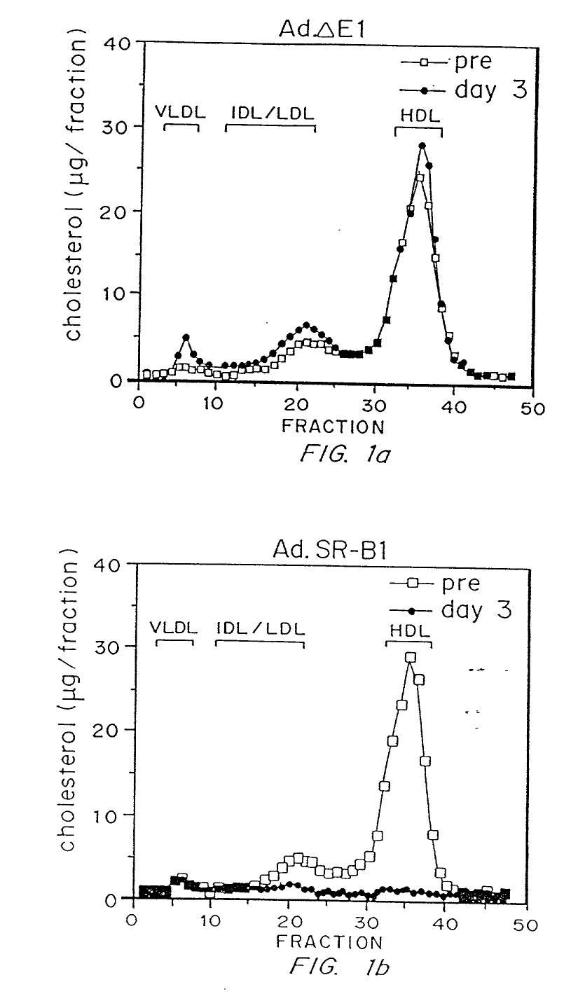

Fast pressure liquid chromatography (FPLC) analysis of plasma was performed to determine specifically the effects of hepatic SR-BI overexpression on the different classes of lipoproteins. FIGS. 1A and 1B (pre-treatment) show the lipoprotein profile of normal C57BL/6 mice, with most cholesterol contained in the HDL fraction, and low or undetectable VLDL and IDL/LDL fractions. Infusion of the control Ad.ΔE1 virus had virtually no effect on the lipoprotein profiles at earlier (FIG. 1A, pretreatment to day 3) or later (FIG. 1C, days 7 to 21) time points, consistent with the absence of changes in total plasma cholesterol and apoAI levels (Table 1). Plasma lipoproteins of SR-BI infused mice, although identical to control mice pre-infusion, showed a large decrease in HDL cholesterol on day 3 (FIG. 1B). This suggests that SR-BI overexpression in liver causes increased uptake of plasma HDL cholesterol, and thus lowers circulating HDL levels. This is consistent with the lower total plasma cholesterol levels on day 3 (Table 1). At later time points, SR-BI levels slowly declined, and HDL cholesterol slowly increased (Figure 1D). In parallel, on days 7 and 10, an increase in both VLDL and IDL/LDL cholesterol were observed, suggesting either increased VLDL secretion by the liver, or a down-regulation of LDL receptors. These changes may occur as a result of increased cholesterol uptake by the liver through HDL-derived cholesterol taken up by SR-BI. The VLDL and IDL/LDL levels decreased to baseline levels by day 21, although HDL cholesterol remained below baseline, suggesting that SR-BI may still be active. The increase in VLDL and IDL/LDL was not seen in all virus preparations.

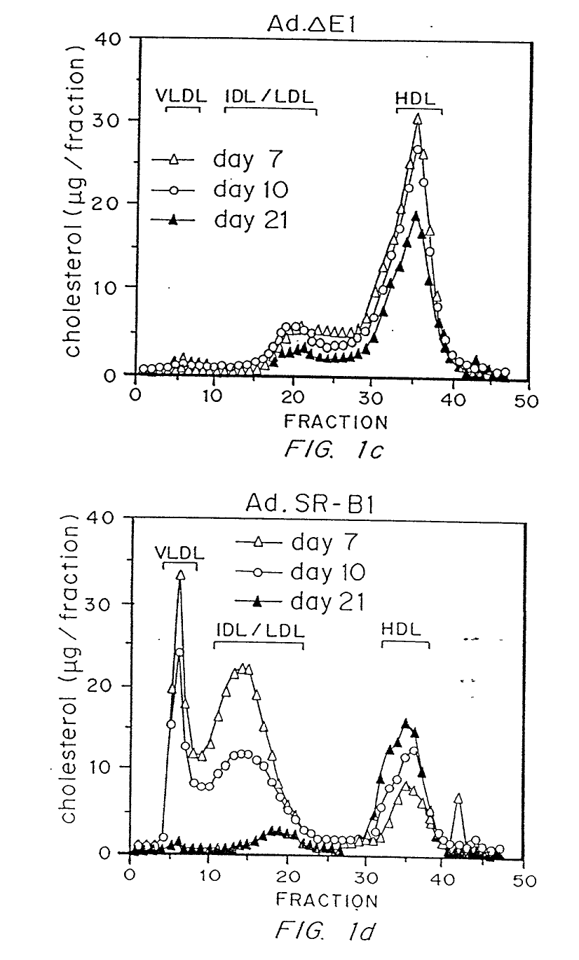

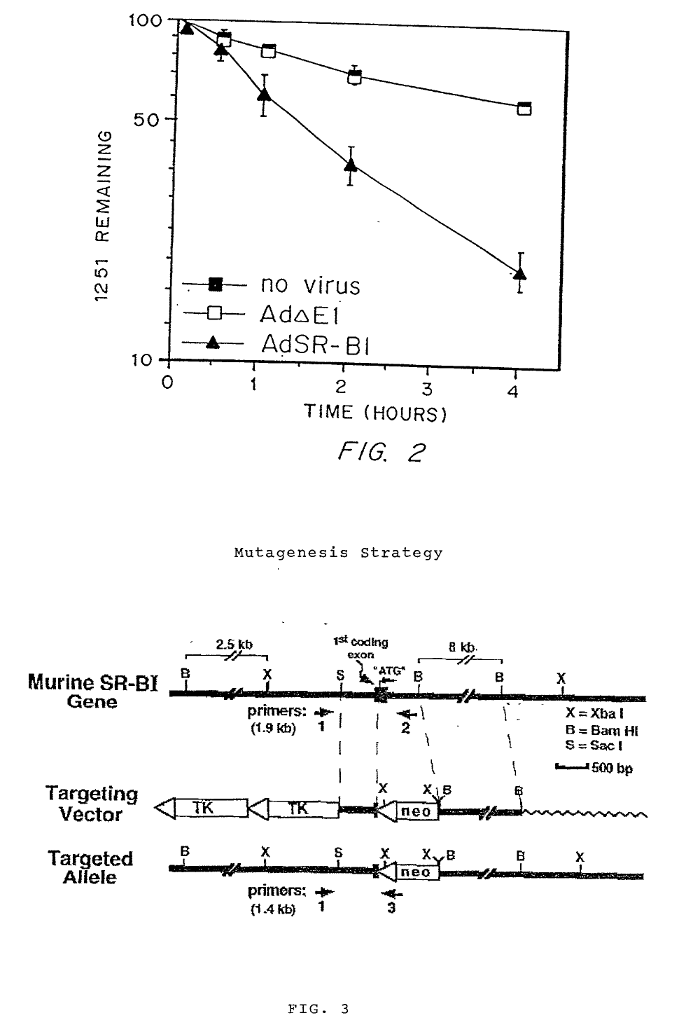

To examine the fate of the HDL particle, an HDL clearance study was performed. Mice were infused with either the control virus Ad.ΔE1, or with Ad.SR-BI. Five days following virus infusion, when transgene expression levels are maximal, mice were infused with ¹²⁵I-labeled HDL, which is labeled in the protein portion (primarily apoAI). Plasma samples were obtained at various time points, and the amount of ¹²⁵I remaining in the plasma was determined. FIG. 2 shows that mice overexpressing SR-BI (triangles) had a faster rate of HDL turnover than either uninfused (closed squares) or control virus infused mice (open squares). This suggests that the remnantHDL particle itself may be degraded, possibly in the kidney, following hepatic SR-BI-mediated uptake of HDL-derived cholesterol.

Unlike LDL cholesterol, HDL-derived cholesterol is believed to be preferentially excreted in bile. Thus, bile excreted from SR-BI overexpressing mice was analyzed for cholesterol, bile salt, and phospholipid content. Four days following infusion of control virus (Ad.ΔE1) or Ad.SR-BI, mice were anesthetized, bile ducts were cannulated, and bile collected for approximately 1 hour to obtain at least 0.1 ml of bile. Table 2 shows that bile from SR-BI mice contained approximately 2-fold more free cholesterol than control mice, while bile salts and phospholipid did not change. This demonstrates that one consequence of increased hepatic uptake of HDL cholesterol is increased cholesterol excretion in bile.

TABLE 2
Bile cholesterol levels.

| | Cholesterol | Bile salts | Phospholipid |
|---|---|---|---|
| | (mM) | (mM) | (mM) |
| no virus | 0.490 ± 0.138 | 20.5 ± 6.4 | 3.95 ± 1.01 |
| Ad.ΔE1 | 0.572 ± 0.132 | 23.2 ± 10.7 | 3.64 ± 1.24 |
| Ad.SR-BI | 1.149 ± 0.358⁸ | 19.7 ± 5.9 | 4.72 ± 1.48 |

n = 8 to 13 for each group
^{a}p < <0.0005 compared to both no virus and Ad.ΔE1 controls

As an surrogate marker of HDL-cholesterol transfer to hepatocytes, mice were injected with DiI-HDL, which are labeled with a fluorescent lipid (DiI). These particles have previously been shown in cell culture to transfer the DiI at a rate comparable to the rate of transfer of the cholesterol ester. Five days after virus infusion, mice were injected with 40 μg of Dil-HDL. Two hours later, mice were anesthetized, perfused, and liver tissues were taken. Fresh-frozen sections of liver from SR-BI overexpressing mice stained strongly with the anti-SR-BI antibody and had high DiI content, as viewed under the fluorescent microscope. In contrast, control mice had low DiI content. Furthermore, in several mice, DiI transfer to bile was measured. Bile from control mice (n=7) had fluorescence intensity ranging from 0.11 to 0.19 (relative units). In contrast, bile from the two SR-BI overexpressing mice in this experiment had fluorescence intensities of 1.13 and 0.93.

Taken together, these data show that hepatic SR-BI overexpression increases uptake of HDL-derived lipid into the liver, and that in turn some of the cholesterol can be excreted in the bile. These data further suggest that inhibition of SR-BI should increase HDL cholesterol blood levels. This is expected to provide a mechanism for decreasing cholesterol secretion into the gall bladder and therefore inhibit gallstone formation.

### EXAMPLE 6

Production and Characterization of Transgenic Animals Which do not Express SR-BI

To determine directly if SR-BI normally plays an important role in HDL metabolism in vivo and to establish an experimental system to examine the role of SR-BI in pathologic states, mice containing a targeted null mutation in the gene encoding SR-BI were generated.

Materials and Methods

Generation of SR-BI Mutant Mice.

SR-BI genomic DNA was isolated from a mouse strain 129 DNA library (Genome Systems, St. Louis, Mo.), and screened by PCR amplification using primer pairs corresponding to the 5′ and 3′ ends of the mSR-BI cDNA. From one clone a 12 kb Xba I fragment containing the first coding exon was identified. A replacement-type targeting vector, containing 0.75 kb and 9 kb short and long homology regions and the pol2sneobpA and herpes simplex virus thymidine kinase (TK) cassettes, was constructed using standard methods. The vector was linearized and 100 μg were transfected by electroporation (240 V, 500 μF) into 112×10⁶ murine D3 embryonic stem cells, which were then plated onto irradiated mouse embryonic fibroblast feeder layers. After G418/gancyclovir positive/negative selection for 7-8 days, 492 of the 5800 surviving colonies were picked and screened by PCR analysis using primers specific for the targeted allele (primer 1 5′-TGAAGGTGGTCTTCAAGAGCAGTCCT-3′ (SEQ ID NO:5); and primer 3 5′-GATTGGGAAGACAATAGCAGGCATGC-3′ (SEQ ID NO:6); all oligonucleotide primers were synthesized by Research Genetics). The presence of the targeted allele (amplification of a 1.4 kb band) was confirmed by Southern blot analysis of Xba I digested genomic DNA using probes that yielded either the predicted 12 kb fragment characteristic of the wild-type allele or the predicted 2.5 kb and 9 kb fragments from the targeted mutant allele. Bam HI digested genomic DNA was also probed with a 0.9 kb fragment derived by Pst I digestion of the neomycin resistance gene cassette to confirm the presence of a single neo gene in the mutant cells. Embryonic stem cell clones containing a disrupted SR-BI allele were injected into C57BL/6 blastocysts, which were implanted into recipient females. The resulting chimeric mice were crossed to C57BL/6 female mice to generate Fl wild-type (srbI^{+/+}) and heterozygous (srb+/^{+/+-}) mice on an identical 129 (agouti)/C57BL/6 background. Fl heterozygotes were crossed to generate F2 wild-type (srbI^{+/+}), heterozygous mutant (srbI^{+/-}) and homozygous mutant (srbI^{+/-}) progeny. The presence of the targeted or wild-type SR-BI alleles in DNA extracted from tail biopsies was detected by PCR amplification using primer 1 in combination with either primer 3 (mutant specific) or primer 2 (wild-type specific; 5′-TATCCTCGGCAGACCTGAGTCGTGT-3′ (SEQ ID NO:7)). Genotypes were confirmed by Southern blot analysis. Mice were housed in microisolator cages and were fed ad libitum a regular rodent chow diet (Prolab 3000, PMI Feeds Inc., St. Louis, Mo.).

Analysis of Animal Tissues:

Samples were obtained from fasted (4-8 hrs) or non-fasted mice that were approximately 8-12 weeks old (Fl generation) or 5-11 weeks old (F2 generation).

Immunoblot Analysis.

Animals were sacrificed and livers and adrenal glands were removed and immediately frozen. Membranes from homogenates were prepared. 50 μg of protein per specimen were analyzed by SDS-polyacrylamide (8%) gel electrophoresis and immunoblotting with chemiluminescence detection as previously described using rabbit antipeptide polyclonal antibodies which specifically recognize either the approximately 82 kDa murine SR-BI protein (anti-mSR-BI⁴⁸⁵) or the approximately 36 kDa ε-COP control cytoplasmic protein (anti-εCOP).

Plasma and Adrenal Cholesterol Analysis.

Plasma total cholesterol (unesterified plus esterified, mg/dl) was measured using an enzymatic kit (Sigma Chemicals, St. Louis, Mo.). Adrenal glands were homogenized as described above. Protein concentrations in the homogenates were measured using the method of Lowry et al. Duplicate samples of homogenates (30-70 μl each) were extracted with 2 ml of hexane/isopropanol (2:1) for 1 h at room temperature, back-washed with 1 ml of water, and phases separated by centrifugation at 800× g for 5 min. The upper organic phase was recovered and evaporated at 37° C. in a Speedvac concentrator and cholesterol was measured in the dried pellet using an enzymatic kit (Sigma). Cholesterol values were corrected based on the recovery of a [³ H]cholesteryl ester internal standard added prior to lipid extraction. Total cholesterol content was expressed as μg of cholesterol/mg total protein.

Lipoprotein Analysis.

Pooled plasma (150 μl total from 2-6 animals) was diluted with an equal volume of elution buffer (154 mM NaCl 1 mM EDTA, pH 8) and subjected to FPLC using two Superose 6 columns (Pharmacia, Piscataway, N.J.) connected in series. Proteins were eluted at 0.25 ml/min. Forty seven fractions (0.5 ml) were collected after the first 14 ml were eluted and total cholesterol in each fraction was determined as described above. Immunoblotting of the FPLC fractions was performed with specific anti-apoA-I, anti-apoA-II or anti-apoE antibodies on independent samples or by sequential labeling of a single membrane to permit simultaneous visualization of all three proteins.

Statistical Analysis.

Results are expressed as the arithmetic mean±standard deviation. The statistical significance of the differences of the mean between groups was evaluated using the Student t test for unpaired comparisons. The χ² test was used for genotype distribution analysis. P values <0.05 are considered to be statistically significant.

Results and Discussion

The SR-BI gene was inactivated in embryonic stem cells by standard homologous recombination methods. The segments replaced in the recombined mutant (“Targeted Allele”) include the entire coding region of the first coding exon (126 bp, 42 amino acids, containing 5′ untranslated sequence, a short N-terminal cytoplasmic domain, and a portion of the N-terminal putative transmembrance domain that probably also functions as an uncleaved leader sequence for insertion into the ER during biogenesis) and an additional 554 bases of the adjacent downstream intron. The mutated locus is expected to encode a transcript which would not be translated or would be translated into non-functional, non-membranous, and presumably unstable, protein. The strategy for the targeted disruption of the SR-BI locus in the mouse is shown in FIG. 3. FIG. 3 is a restriction map of the genomic DNA surrounding the first coding exon of the murine gene encoding SR-BI. The targeting vector and the predicted structure of the targeted (mutant) allele are shown and described in the text. The locations of the sequences for the PCR primers used to specifically detect either the wild-type (primers 1 and 2) or targeted mutant (primers 1 and 3) alleles are indicated along with the predicted PCR product lengths. Abbreviations: TK, herpes simplex thymidine kinase; neo, pol2sneobpA expression cassette, X, Xba I; B, Bam, HI; S, Sac I; “ATG”, codon for the initiator methionine. Two sets of primer pairs specific for the wild-type (primers 1 and 2) or targeted mutant (primers 1 and 3) alleles were used to screen genomic DNA by PCR as described in heterozygous and F2 homozygous mutant animals are shown. Immunoblot analysis of hepatic membranes (50 μg protein/lane) from unfasted wild-type (F1 and F2 generations), heterozygous (F1 and F2 generations) and homozygous mutant (F2 generation) male mice were performed using polyclonal antipeptide antibodies to SR-BI (approximately 82 kDa, top) or the internal control ε-COP (approximately 36 kDa). Essentially identical results were obtained using specimens from female mice) confirmation of the expected null mutation by PCR.

Three independently derived embryonic stem cell clones containing the targeted allele were injected into C57BL/6 blastocysts and two produced 24 male chimeras, of which 11 gave germ line transmission of the targeted SR-BI allele when crossed to c57BL/6 females. F1 offspring were either homozygous (+/+) for the wild type allele or heterozygous (+/−) with both mutant and wild-type PCR products. Fl heterozygotes should be isogenic with the F1 wild-type controls except at the SR-BI locus. Wild-type, heterozygous and homozygous mutant F2 generation offspring, whose phenotypes are subject to genetic background variability, were generated from F1 intercrosses. In the F2 progeny analyzed to date (n=317), the observed ratios of wild-type heterozygous mutant homozygous mutant offspring were 1.0:1.7:0.5, values significantly different from the expected Mendelian ratio of 1:2:1 (p=0.003). Thus, there may be partially penetrant effects of the mutation either on neonatal survival or on embryonic development, which would be consistent with the distribution of SR-BI on the maternal surfaces of cells in the placenta and yolk sac during embryonic development.

All of the mutants looked normal (weight, general appearance and behavior) and the males were fertile. No offspring from female homozygous mutants have been obtained following multiple attempts to do so, indicating a substantial, and possibly complete, decrease in fertility in these females. Immunoblot analysis of liver membranes from F1 (+/+, +/−) and F2 (+/+, +/−, −/−) mice using anti-peptide antibodies which recognize the C-terminus of the SR-BI protein (anti-mSR-BI⁴⁹⁵), or a segment of the putative extracellular loop (anti-mSR-BI²³⁰), revealed that there was about half as much mSR-BI protein in the heterozygous mutants as in the wild-type controls and no detectable SR-BI in the homozygous mutants. No fragment or other variants of the full-length protein were detected in any of the samples. In contrast, no significant differences were observed in the levels of the control protein, ε-COP. Similar results were observed using adrenal tissue. Thus, the mutated SR-BI gene is a functionally null allele.

To determine how decreased SR-BI protein expression influenced lipoprotein metabolism, the plasma cholesterol levels in male and female wild-type and mutant mice were compared. Because there were no statistically significant differences between the data from animals derived from the two independent embryonic stem cell clones, data from these two independent sets of animals were pooled. Relative to wild-type controls there were statistically significant increases in the plasma total cholesterol concentrations of approximately 30-40% in F1 and F2 heterozygotes and 2.2-fold in F2 homozygous mutants. In contrast to the increased plasma cholesterol in the mutants, there was no statistically significant change in the levels of plasma apoA-I. These findings are consistent with the suggestion that hepatic SR-BI plays a key role in selective removal of cholesterol from circulating HDL-lower levels of hepatic SR-BI were expected to increase plasma HDL cholesterol but not directly alter apoA-I levels.

To determine if the elevated levels of plasma cholesterol in the mutants were due to changes in HDL, pooled plasma samples from F1 male and female and F2 male animals were subjected to FPLC and the total cholesterol content as well as the relative amounts of apoA-I, apoA-II and apoE in each fraction were measured. The results are shown in FIG. 4. For wild-type mice (srbI^{+/+}) most of the cholesterol (panel A open squares), apoA-I and apoA-II were in the HDL fraction, with small or undetectable amounts in the VLDL and IDL/LDL fractions. There was an apparently low level of apoE which both co-migrated with the HDL (centered around fraction 35) and with a small cholesterol peak in the IDL/LDL region (around fraction 20, very faint bands). The cholesterol and apolipoprotein profiles of the heterozygous mutants were similar to those of the wild-type controls, except that there was an increase in the amount of cholesterol in the HDL fractions and there was a tendency of the HDL peak (cholesterol and/or apolipoproteins) to be broader than that of wild-type and shifted slightly to the left (lower fraction number, greater apparent size), which may represent large HDL particles. This suggested that there might be a difference in the average sizes of the HDL particles due to the inactivation of one of the SR-BI alleles; however, this shift was not observed in all specimens. In the F2 homozygous mutant animals (srbI^{-/-}) the cholesterol was found in a large, somewhat heterogeneous peak in the HDL range, but shifted to the left (larger apparent size) of the wild-type HDL peak. The amount of cholesterol in the IDL/LDL fraction (around fraction 20) varied between samples.

Combined immunoblot analysis of fractions 23-28 from the chromatograms shown in FIG. 4 were performed with polyclonal antibodies to apoe, apoA-I and apoAII. Additional analysis of these and independent chromatograms established that there were no additional peaks containing apoA-I in fractions containing larger lipoproteins (fractions 1-22) and that the only other peak containing a small amount of apoE was in fraction 6, which corresponds to VLDL. The distributions of apoA-I and apoA-II were similar to that of cholesterol, although, unlike the case for apoA-I there was a notable reduction in the amount of apoA-II relative to that seen in wild type and heterozygous mutant animals. Conversely, in the homozygous mutants there was a substantial increase in the amount of apoE, whose distribution profile (larger particles, centered around fractions 26-28) differed from, but overlapped, those of apoA-I and apoA-II.

These results with the mutant animals, in which the changes in SR-BI expression are in the physiologic range, are complementary to and consistent with the observation that transient adenovirus-mediated hepatic SR-BI overexpression results in dramatically decreased levels of HDL cholesterol and increased delivery of HDL-associated lipid to hepatocytes and the bile. In rodents, most of the plasma HDL cholesterol appears to be removed by the liver via selective uptake and the liver appears to be the site of the highest total amount of SR-BI protein expression. It seems likely that buildup of large, cholesterol-enriched lipoprotein particles in the circulation of SR-BI mutants was primarily due to decreased hepatic selective HDL cholesterol uptake. Thus, it appears that murine plasma HDL cholesterol levels are particularly sensitive to physiologically relevant changes in the levels of hepatic SR-BI protein expression (e.g., approximately 50% reduction in heterozygotes). The effect of the null mutation in SR-BI on total plasma cholesterol levels was quantitatively similar to that of a null mutation in the LDL receptor. For both sets of mutants, total plasma cholesterol levels were approximately 36% above wild-type controls for heterozygotes and approximately 114% for homozygotes. It is important to emphasize that while the magnitudes of the effects on total plasma cholesterol of these distinct mutations (SR-BI vs. LDL receptor) are similar, the mechanistic consequences on lipoprotein metabolism (e.g., effects on the various lipoproteins) differ.

In addition to playing an important role in regulating plasma HDL cholesterol, SR-BI has been implicated in the delivery of HDL cholesterol to the adrenal gland and other steroidogenic tissues, both for the accumulation of esterified cholesterol stores and for steroid hormone synthesis. To examine this, the cholesterol content of adrenal glands in mutant and wild-type mice was measured. The results are shown in Table 3. As predicted, cholesterol stores in the adrenal gland dropped substantially in the heterozygous and homozygous mutants to 58% and 28% of control, respectively. It was also noted that the color of intact adrenal glands from homozygous mutants was brownish-red while that of wild-type and heterozygous animals was light yellow and, in preliminary studies, a dramatic decrease in oil red O staining of the adrenal cortex was observed in the homozygous mutants relative to the wild-type mice. Thus, the total cholesterol content, color and oil red O staining characteristics of the adrenal glands in SR-BI homozygous mutants resembled those in their cholesterol-depleted counterparts in other murine mutants, including null mutants in the SR-BI ligand apoA-I. This similarity with apoA-I knockouts is consistent with the possibility that the reduction in adrenal cholesterol in the SR-BI homozygotes is a direct consequence of the loss of the key receptor for selective lipid uptake. Recent antibody blocking experiments have provided additional support for a major role of mSR-BI in delivering HDL cholesterol to cultured adrenocortical cells for steroidogenesis. Based on the tissue distribution and hormonal regulation of SR-BI protein expression and the phenotypes of apoA-I knockouts, it seems likely that there would also be reductions in cholesterol stores in other steroidogenic tissues (e.g., ovary, testes) in SR-BI homozygous mutants. Adrenal cholesterol deficiency in both the apoA-I and SR-BI homozygous mutants also suggests that LDL receptors in the mouse, in which there normally is little LDL in the plasma, do not normally contribute significantly to murine adrenal cholesterol accumulation.

TABLE 3
EFFECTS OF DISRUPTION OF THE GENE ENCODING SR-BI ON PLASMA TOTAL CHOLESTEROL
AND APO A-I CONCENTRATIONS, AND ADRENAL GLAND TOTAL CHOLESTEROL CONTENT IN
WILD-TYPE (srbI^{+/+}), AND HETEROZYGOUS (srbI^{+/+}),
AND HOMOZYGOUS (srbI^{-/-}) MUTANT MICE.

| | | F1 Generation | | | | F2 Generation | | | |
|---|---|---|---|---|---|---|---|---|---|
| | | Plasma Total | | Plasma Total | | | | Adrenal Gland Total | |
| | | Cholesterol | | Cholesterol | | Plasma ApoA-1 | | Cholesterol | |
| srbI | | | % of | | % of | | % of | | % of |
| genotype | gender | mg/d1 | control | mg/d1 | control | mg/d1 | control | μ/mg protein | control |
| | male | 93 ± 8 (29) | 100 | 99 ± 12 (18) | 100 | — | — | — | — |
| +/+ | female | 80 ± 7 (13) | 100 | 94 ± 20 (27) | 100 | — | — | — | — |
| | both | 89 ± 10 (42) | 100 | 96 ± 17 (45) | 100 | 25 ± 3 (10) | 100 | 128 ± 28 (5) | 100 |
| | male | 126 ± 10 (21) | 100 | 137 ± 21 (29) | 100 | — | — | — | — |
| +/− | female | 112 ± 9 (23) | 100 | 118 ± 9 (49) | 112 | — | — | — | — |
| | both | 126 ± 12 (44) | 134 | 126 ± 22 (78) | 131 | 28 ± 2 (12) | 112 | 74 ± 18 (6) | 58 |
| | male | — | — | 220 ± 41 (10) | 222 | — | — | — | — |
| −/− | female | — | — | 209 ± 32 (7) | 222 | — | — | — | — |
| | both | — | — | 216 ± 37 (17) | 225 | 27 ± 3 (11) | | 36 ± 7 (5) | 28 |

Values for F1 generation represent mean ± standard deviation. Values for F2 generation in parenthesis the numbers of animals analyzed.
Values for plasma total cholesterol determined with an Autoanalyzer and human apoA−I standards.
F1 generation animals were not fasted. F2 generation animals were not fasted prior to analysis of adrenal gland cholesterol levels but were fasted for 4-8 h
prior to analysis of plasma.

### EXAMPLE 7

### Reproductive Studies with SR-BI Knockout Mice

The female homozygous knockout mice are infertile. Several studies were conducted to determine why. These animal do exhibit estrus and ovulate. However, examination of the eggs shows them not to be viable, and to be extremely fragil, with eggs isolated after mating, at the one, two or four cell stage dying with 24 hours.

Additional studies have been conducted to look at implantation. Females were mated with vasectomized males, then implanted with normal wild type embryos. At mid-gestation, the majority of wild type controls show implantation; none of the knockout females show implantation. Preliminary studies indicate that the homozygous females have a defect in the decidual reaction.

### EXAMPLE 8

### Inhibition of Steroid Production by Adrenal Cells Using an Anti-SR-BI Antibody

In this study, the function of R-BI in steroidogenic cells was tested directly with antibody raised against a portion of the extracellular domain of the protein. The results establish that SR-BI serves as the major route for the selective uptake of HDL CE and for the delivery of HDL cholesterol to the steroidogenic pathway in cultured adrenal cells.

Material and Methods

Preparation of Antibodies to mSR-BI.

Rabbit polyclonal antibodies were raised to a glutathione-S transferase (GST) fusion protein containing mSR-BI amino acid residues 174-356. This corresponds to approximately 45% of the putative extracellular domain (amino acid residues 33-439) of the receptor. For this purpose, oligonucleotides (sense XmaI primer, 5-GATGGCCCGGGCCGCACAGT TGGTGAGATCC-3 (SEQ ID NO:8), and antisense XhoI primer, 5-GGATAGCCCTCGAGTTCTGACAACACAGGGTCGGC-3 (SEQ ID NO:9), were used to PCR amplify bases 520-1,068 from the ORF of mSR-BI under the following conditions: 2.5 mM MgCl₂, 0.01% gelatin, 62.5 μM dNTPs, 0.5 μM sense XmaI primer, 0.5 μM antisense XhoI primer, 20 ng pcDNA3-mSR-BI, 1? PCR reaction buffer, and 1 unit Taq DNA polymerase (Boehringer Mannheim). PCR reactions were carried out with a 1 cycle denaturation program (95° C. for 5 min), a 35 cycle amplification program (95° C. for 45 sec, 58° C. for 45 sec, and 72° C. for 60 sec), and a 1 cycle extension program (72° C. for 7 min). The PCR product and pGEX-4T-1 (Pharmacia) were cut with XhoI and XmaI (New England Biolabs), gel purified, and ligated overnight. Ligation products were transformed into Max efficiency DH5 competent cells (GIBCO/BRL) and selected on Luria broth plates containing 100 μg/ml ampicillin. The desired plasmid, pGEX-4T-1-mSR-BI EC, was identified by restriction enzyme mapping, and the entire mSR-BI coding region and cloning junctions were sequenced.

For purification of the fusion protein, pGEX-4T-1-mSR-BI EC was transformed into TG-1 cells, and GST-mSR-BI EC fusion protein was isolated by a modified version of the protocol of Smith and Johnson ((1988) Gene 67, 31-40; Koff, et al. (1992) Science 257, 1689-1694). Following induction with isopropylthiogalactoside, cells were lysed by sonication in 10 mM Tris-HCl (pH 7.4), 100 mM NaCl, 1 mM MgCl2, 5 mM DTT, 10 μg/ml aprotinin, 1 μg/ml leupeptin, 1 μg/ml pepstatin, and 0.2 mM phenylmethylsulfonyl fluoride. The lysate was centrifuged for 10 min at 10,000× g, and the pellet containing the fusion protein was washed twice by resuspension in 0.2 M Tris·HCl (pH 8), 0.5 M NaCl, 5 mM DTT (TN buffer), followed by centrifugation as above. The pellet was extracted with 8 M urea/5 mM DTT for 1-3 hr at 4° C., dialyzed against TN buffer, cleared by centrifugation, and incubated with glutathione agarose (Sigma) for 1-2 hr at 4° C. The glutathione agarose was washed with TN buffer, and the fusion protein was eluted in TN buffer containing 20 mM glutathione. Two male New Zealand White rabbits (Rb355 and Rb356) were immunized with 300 μg of fusion protein in Freund's complete adjuvant and boosted with 150 μg of fusion protein in incomplete Freund's adjuvant at weeks 2, 3, and 7. Thereafter, rabbits were boosted three times at monthly intervals with an SDS/10% polyacrylamide gel slice containing 250 μg of the SR-BI fragment that had been cleaved from the fusion protein by thrombin digestion. Ten days after the last boost, rabbits were exsanguinated, and IgG was prepared by chromatography on protein A-agarose (Bio-Rad) (Harlow & Lane (1988) Antibodies: A Laboratory Manual (Cold Spring Harbor Lab. Press, Plainview, N.Y.)). Control or nonimmune IgG was prepared from two rabbits that had not been immunized. Prior to incubation with cells, IgG was dialyzed against 25 mM ammonium bicarbonate (pH 7.4), lyophilized, reconstituted in F10 serum-free medium, and cleared by centrifugation. Protein concentration was determined according to Lowry et al. ((1951) J. Biol. Chem. 193, 265-275).

Characterization of Rb355 and Rb356 mSR-BI EC IgG by Western Blotting.

Postnuclear supernatant was isolated from ldlA[mSR-BI] and Y1-BS1 cells as described (Acton, et al. (1996) Science 271, 518-520[Abstract]; Rigotti, et al. (1996) J. Biol. Chem. 271, 33545-33549), except that lysis buffer also contained 10 μg/ml aprotinin, 1 μg/ml leupeptin, 1 μg/ml pepstatin, and 0.2 mM phenylmethylsulfonyl fluoride. Proteins (20 μg) were resolved on an SDS/8% PAGE gel, transferred to a nitrocellulose membrane, and probed with IgG as described (Rigotti, et al. 1996). Antibody binding was visualized by chemiluminescence detection (Amersham) using Reflection autoradiography film (NEN/Dupont).

Preparation of [¹²⁵I]Dilactitol Tyramine-[³H]Cholesteryl Oleolyl Ether hHDL3 ([¹²⁵I,³H]hHDL3), [³H]Cholesteryl Oleate hHDL3 ([³H] hHDL3), and [³H]Cholesteryl Oleate Recombinant (r) HDL ([³H] rHDL).

Human (h) HDL3 (1.125 g/ml <<1.210 g/ml) labeled with [¹²⁵I]dilactitol tyramine and [³H]cholesteryl oleolyl ether was prepared as described by Azhar, et al. (1989) J. Lipid Res. 30, 1799-181018. The specific activity of the [¹²⁵I,³H]hHDL3 ranged from 46-70 dpm/ng protein for ¹²⁵I and from 6-28 dpm/ng protein for ³H. The specific activity of [³H]hHDL3, prepared as described by Azhar, et al. (1990) Biochim. Biophys. Acta 1047, 148-169; and Azhar, et al. (1989), ranged from 3-5 dpm/ng protein. The specific activity of [³H]rHDL, prepared as described (Reaven, et al. (1995) J. Lipid Res. 36,1602-161721) was 60 dpm/ng protein.

Determination of HDL Cell Association, Selective CE Uptake, and Apolipoprotein Degradation.

Y1-BS1 murine adrenocortical cells (Schimmer, B. P. (1981) Functionally Differentiated Cell Lines (Liss, N.Y.), pp. 61-92) were maintained and experiments were performed in a 37° C. humidified 95% air/5% CO₂ incubator as described by Rigotti, et al., 1996. For all experiments, 6-well plates (Costar), which had been treated with 100 μg/ml poly D-lysine (Becton Dickinson), were seeded with Y1-BS1 cells at a density of 1.5×10⁶ cells per well. After 48 hr, medium was removed and replaced with 1.5 ml Ham's F-10 complete medium plus or minus 100 nM Cortrosyn (Organon), a synthetic 1-24adrenocorticotropic hormone (ACTH) analogue. After 24 hr, medium was replaced with serum-free Ham's F-10 medium lacking ACTH and containing no additions or 6 mg/ml IgG consisting of varying proportions of nonimmune IgG and anti-mSR-BI IgG. After a 1-hr preincubation, [¹²⁵I,³H]hHDL3 was added at 10 μg protein/ml (except where indicated), and the incubation was continued for 2 or 4 hr. Cells were washed three times with 0.1% BSA in PBS (pH 7.4), one time with PBS (pH 7.4), lysed with 1.25 ml 0.1 N NaOH, and passed five times through a 28.5-gauge needle. The lysate was then processed to determine trichloroacetic acid soluble and insoluble ¹²⁵I radioactivity and organic solvent-extractable ³H radioactivity as described by Azhar 1989, 1990. Trichloroacetic acid insoluble ¹²⁵I radioactivity represents cell-associated HDL apolipoprotein, which is the sum of cell surface bound apolipoprotein and endocytosed apolipoprotein that is not yet degraded. Trichloroacetic acid soluble ¹²⁵I radioactivity represents endocytosed and degraded apolipoprotein that is trapped in lysosomes due to the dilactitol tyramine label (Azhar 1989; Glass, et al. (1983) J. Biol. Chem. 258, 7161-7167). The sum of the ¹²⁵I-degraded and 125I cell-associated undegraded apolipoprotein expressed as CE equivalents was subtracted from the CE measured as extractable ³H radioactivity to give the selective uptake of HDL CE . Values for these parameters are expressed as nanograms of HDL cholesterol/mg cell protein. The HDL concentration dependence for each of these parameters was modeled by a simple binding isotherm composed of a high-affinity saturable process and a low-affinity nonsaturable process: where Pₜₒₜₐₗ is the measured parameter, [Pₘₐₓ] is the high-affinity parameter at saturating levels of HDL, K_{HA} is the apparent high-affinity Kₘ, and C is the slope of the low-affinity nonsaturable process. For each parameter, Ptotal was resolved into high- and low-affinity components by determining C and subtracting C [HDL] from Pₜₒₜₐₗ to generate the curve for the high-affinity HDL concentration dependence.

Determination of [³H] Steroid Production.

Y1-BS1 cells were preincubated as above with or without 6 mg/ml IgG for 1 hr prior to addition of [³H]hHDL3 at 25 μg protein/ml or [³H]rHDL at 5 μg protein/ml. The incubation was continued for 24 hr in the presence of the indicated IgG and 100 nM 1-24 ACTH. Medium was removed, a [¹⁴C]progesterone recovery standard (New England Nuclear) was added, and the sample was extracted with CH₂Cl₂ as described by Cheng & Kowal (1988) J. Chromatogr. 432, 302-307. Steroids were separated on a Brownlee reverse-phase C18 column (OD-300, Aquapore ODS, 25 cm×4.6 mm) in a mobile phase of methanol: acetonitrile: water (11:45:44), and the peaks corresponding to 11,20-dihydroxy-4-pregnene-3-one, 11-hydroxyprogesterone, and progesterone were collected and counted by liquid scintillation spectrometry. Measured values for [³H] steroids were corrected for recovery losses and normalized for cell protein. Values for samples incubated with IgG were expressed as a percentage of the control samples with no IgG. Control values for [³H] steroid secretion with several preparations of [³H]hHDL3 ranged from 19,000-34,000 dpm/mg cell protein. Control values for samples incubated with [³H]rHDL were 173,000 dpm/mg cell protein.

Determination of DiI-HDL Uptake into ldlA[mSR-BI] Cells.

ldlA[mSR-BI] cells or IdlA cells (Acton, et al. (1996) Science 271, 518-520) were plated at a density of 1.5×10⁴ per well in 24-well plates in 1 ml Ham's F-12 complete media (5% heat-inactivated fetal bovine serum/2 mM L-glutamine/50 units/ml penicillin/50 μg/ml streptomyocin, either with or without 0.5 mg/ml G418, respectively). After an overnight incubation at 37° C. in a humidified 95% air/5% CO₂ incubator, the medium was replaced with 1 ml of DMEM/F-12 serum-free medium (2 mM L-glutamine/50 units/ml penicillin/50 μg/ml streptomyocin). After 24 hr, the medium was removed, and the cells were washed with 0.5 ml DMEM/F-12 serum-free medium. Each well-received 0.2 ml of DMEM/F-12 serum-free media supplemented with or without 6 mg/ml IgG. After a 2-hr incubation at 37° C., DiI-labeled HDL (Acton 1996) was added to 10 μg protein/ml with or without unlabeled HDL at 400 μg protein/ml, and the incubation was continued for 2 hr at 37° C. After washing two times with PBS, cells were removed by trypsin treatment for 3 min followed by quenching with new-born-calf lipoprotein-deficient serum. Fluorescence intensities were measured on a Becton Dickinson FACStar Plus flow cytometer. DiI was excited with 100 mW of 514 nm light from a Coherent Innova 90-5 argon ion laser. Emitted light was collected using a 575 DF/26 filter. Note that for this experiment (using ldlA[mSR-BI] cells) the ratios of the amounts of antibody per cell were much higher than for those for experiments carried out with Y1-BS1 cells. These differences appear to account for differences in apparent KI values measured for antibody inhibition for the two cell types.

Results and Discussion

Characterization of Anti-mSR-BI Antibody.

A polyclonal antibody was raised to a proposed extracellular domain of mSR-BI (Rigotti, et al. (1997) Curr. Opin. Lipidol. 8, 181-188) with the aim of interfering with HDL binding or otherwise disrupting SR-BI function. The Western blots show that 355 and 356 anti-mSR-BI IgG recognized an 82-kDa band in the postnuclear supernatant of ldlA[mSR-BI] cells, a stably transfected Chinese hamster ovary cell line expressing mSR-BI. The mobility of this band was identical to that of mSR-BI detected by a previously characterized anti-peptide antibody directed against the C terminus of the protein. In addition to the 82-kDa mSR-BI band, both 355 and 356 anti-mSR-BI IgG detected low levels of background bands, which were present at comparable levels in ldlA[mSR-BI] and Y1-BS1 cells and in untransfected ldlA cells. The background bands were not related to mSR-BI expression in transfected cells and were not altered by ACTH treatment in Y1-BS1 cells, indicating no functional relationship to the selective uptake process. Neither preimmune IgG nor nonimmune IgG from two other rabbits detected the SR-BI band. In addition, 356 anti-mSR-BI did not detect the SR-BI band in nontransfected ldlA cells. Both 355 and 356 anti-mSR-BI IgG recognized SR-BI in Y1-BS1 adrenocortical cells and readily detected the induction of SR-BI expression when these cells were treated with ACTH. Neither preimmue IgG nor nonimmune IgG recognized SR-BI in Y1-BS1 cell. From these results, it was concluded that 355 and 356 anti-mSR-BI IgG specifically recognized mSR-BI with 356 anti-SR-BI having a significantly higher titer. To evaluate possible immunoreactivity with the related class B scavenger receptor CD36 (Acton, et al. (1994) J. Biol. Chem. 269, 21003-21009), 356 anti-mSR-BI was tested by Western blot analysis with extracts from cells overexpressing rat CD36 and with the entire extracellular domain of CD36 expressed by means of a baculovirus vector. Postnuclear supernatant (20 μg protein) from ldlA[mSR-BI] cells, and Y1-BS1 cells treated without or with 1-24ACTH were separated by SDS/8% PAGE and transferred to nitrocellulose membranes. The membranes were incubated overnight at 4° C. in the presence of either SWB1 nonimmune IgG, SWB2 nonimmune IgG, 355 anti-mSR-BI IgG, or 356 anti-mSR-BI IgG at 4 μg/ml. IgG binding was visualized by enhanced chemiluminescence. No immunoreactivity with CD36 was detected.

Anti-mSR-BI IgG Inhibits DiI-HDL Uptake by ldlA[mSR-BI] Cells.

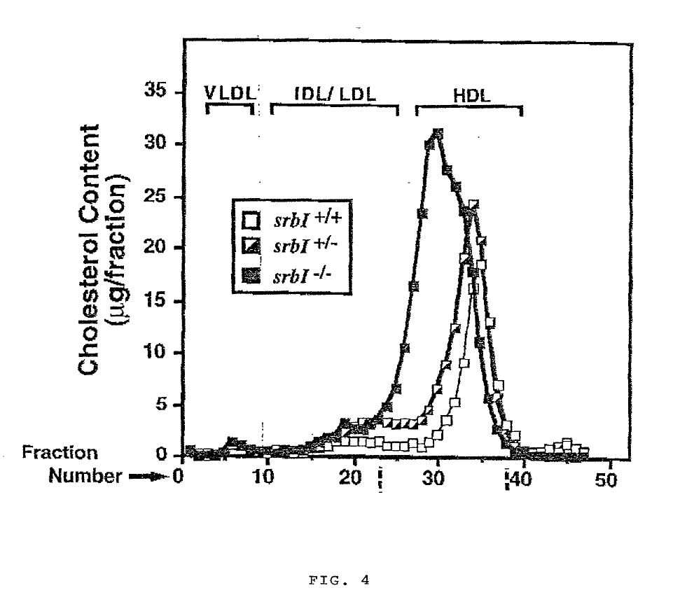

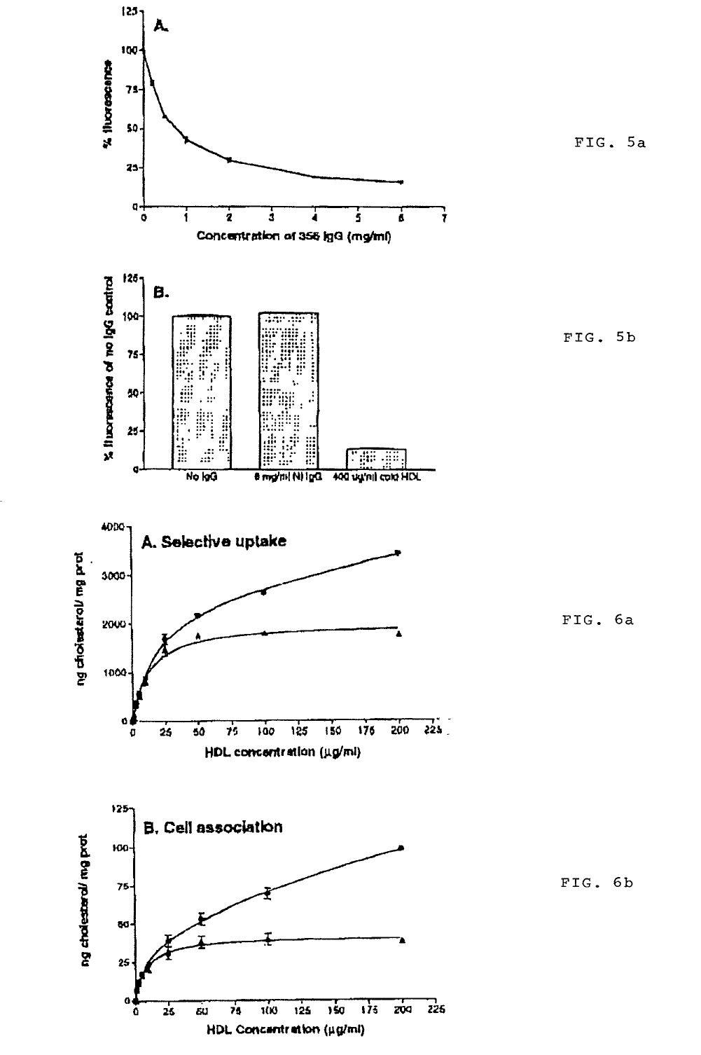

As demonstrated in Example 1, mSR-BI mediates selective uptake of the fluorescent lipid, DiI, from DiI-HDL. To test the ability of the 356 antibody to disrupt SR-BI function, ldlA[mSR-BI] cells, which had been preincubated with increasing concentrations of 356 anti-SR-BI IgG, were exposed to DiI-HDL (10 μg protein/ml), and the accumulation of DiI was measured by flow cytometry. The total IgG concentration in the incubation medium was held constant at 6 mg/ml and the proportion of 356 anti-SR-BI IgG and nonimmune IgG was varied. ldlA[mSR-BI] cells were incubated for 2 hr with DiI-HDL (10 μg protein/ml) in medium containing between 0 and 6 mg/ml 356 anti-mSR-BI IgG and complementary amounts of nonimmune IgG to give a final IgG concentration of 6 mg/ml. Cells were then washed and processed for fluorescence determination by flow cytometry as described. Samples containing 6 mg/ml nonimmune IgG were taken as the 100% control value (arbitrary scale). The results are shown in FIG. 5A. The uptake of DiI-HDL in the presence of no IgG (100% value) is shown in comparison with cells incubated with 6 mg/ml nonimmune IgG or with excess unlabeled in Figure 5B. 356 anti-mSR-BI IgG inhibited the uptake of DiI-HDL in a dose-dependent manner, reaching 85% inhibition at the highest concentration tested. Because a similar inhibition was produced with excess unlabeled HDL (400 μg protein/ml), the 356 antibody appears to have blocked most of the high-affinity interactions between HDL and the ldlA[mSR-BI] cells. In addition, comparison of DiI-HDL uptake by ldlA[mSR-BI] cells incubated with or without 6 mg/ml nonimmune IgG showed that IgG alone had no effect on DiI uptake. These results indicate that 356 anti-mSR-BI IgG effectively disrupts mSR-BI-mediated selective lipid uptake.

Anti-mSR-BI IgG Inhibits HDL-Selective CE Uptake and Cell Association of HDL in Y1-BS1 Cells.

To examine the effects of the anti-mSR-BI antibody on the selective uptake of HDL CE, ACTH-treated Y1-BS1 cells were incubated with [¹²⁵I,³H]hHDL3, which contains [³IH]cholesteryl oleolyl ether as a tracer for CE and [¹²⁵I]dilactitol tyramine-labeled apoproteins as a tracer for cell-associated apolipoprotein, as well as apolipoprotein degraded in lysosomes. Selective CE uptake and cell association is dependent on HDL concentration. In each case, the experimentally measured values show an HDL concentration dependence indicative of both high- and low-affinity components. Based on the assumption that SR-BI would reflect the high-affinity component, antibody inhibition experiments were carried out at 10 μg protein/ml HDL, a concentration at which 90% of the selective uptake is due to the high-affinity component. HDL cholesterol taken up through the selective uptake pathway exceeded by a factor of 40 the HDL cholesterol accounted for by cell association of HDL apolipoprotein, as shown by a comparison of FIGS. 6A and 6B. The amount of HDL cholesterol accounted for by degraded apolipoprotein was even less (1% of the selective CE uptake), illustrating that there was very little HDL apolipoprotein degradation.

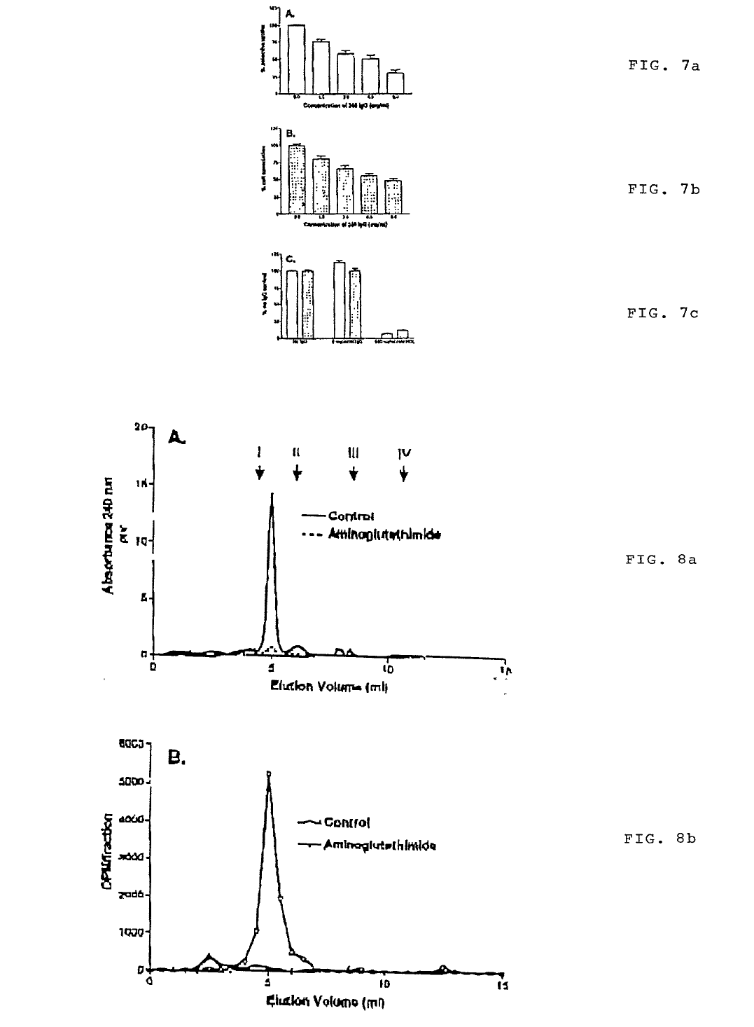

FIG. 7A shows that 356 anti-mSR-BI IgG caused a dose-dependent decrease in HDL-selective CE uptake, which reached 70% inhibition of the total uptake (high plus low affinity) at the highest IgG concentration tested. In similar experiments with the lower titer anti-mSR-BI antibody, the maximum dose-dependent inhibition at 6 mg/ml 355 anti-mSR-BI IgG was 31%. As shown in FIG. 7C, the addition of 6 mg/ml nonimmune IgG alone had no effect on HDL-selective CE uptake (open bars). In addition, when the Y1-BS1 cells were exposed to both [125I,3H]hHDL3 (10 μg protein/ml) and a 50-fold excess of unlabeled HDL3, total selective uptake was reduced to 7% of control (FIG. 7C). This result indicates that approximately 90% of the selective uptake at 10 μg/ml HDL corresponded to the high-affinity component as predicted by the analysis in FIG. 6. Thus, 356 anti-mSR-BI at a concentration of 6 mg/ml inhibited 75% of the high-affinity selective CE uptake. These data indicate that SR-BI is responsible for most of the high-affinity HDL-selective CE uptake in cultured adrenocortical cells.

FIG. 7B shows that 356 anti-mSR-BI IgG caused a dose-dependent decrease in cell association of HDL, which reached 50% inhibition at the highest IgG concentration tested. Nonimmune IgG alone had no effect on cell association of HDL, and excess unlabeled HDL reduced cell association of HDL by 85% (FIG. 7C). Thus, approximately 57% of the high-affinity cell association of HDL was blocked by 356 anti-SR-BI IgG. Because most of the cell association of HDL is believed to reflect cell surface bound lipoprotein particles, this result suggests that 356 anti-mSR-BI inhibits HDL-selective CE uptake primarily by interfering with HDL binding to SR-BI. Interestingly, at all antibody or HDL concentrations examined, the inhibition of binding was consistently less than the inhibition of selective uptake. This result may indicate either that there are multiple sites on SR-BI for HDL binding or that HDL may bind with high affinity to cell surface sites other than SR-BI.

Anti-mSR-BI IgG Inhibits the Delivery of HDL CE to the Steroidogenic Pathway.

Having established that the anti-mSR-BI IgG blocks HDL binding to SR-BI and SR-BI-mediated selective lipid uptake, the blocking antibody was used to determine whether SR-BI is directly involved in providing substrate cholesterol to the steroidogenic pathway. In the presence and absence of the antibody, Y1-BS1 cells were exposed to [³H]hHDL3 particles containing [³H]cholesteryl oleate, and the types and amounts of the secreted radiolabeled steroids were determined using HPLC. The HPLC absorbance profile in FIG. 8A shows that, as previously reported (Cheng & Kowal (1988); Kowal & Fieldler (1968) Arch. Biochem. Biophys. 128,406-421), there is one major steroid product of Y1 cells, 11,20-dihydroxy-4-pregnene-3-one (elution at 5 ml), as well as minor amounts of others, including 11-hydroxyprogesterone (elution at 6 ml). The profile of radiolabeled steroids produced by cells incubated with [³H]hHDL3 (FIG. 8B) was coincident with the absorbance profile. Both the mass of secreted steroids (FIG. 8A) and the radioactive steroids (FIG. 8B) were eliminated when the cells were incubated with aminoglutethimide, an inhibitor of steroid production that inhibits the p450 side-chain cleavage enzyme (Kowal (1969) Endocrinology 85, 270-279) (FIG. 8). Using this assay, radiolabeled steroids secreted by Y1-BS1 cells in response to 25 μg protein/ml [³H]hHDL3 were quantified in cells incubated with no antibody or with 6 mg/ml of either nonimmune IgG or 356 anti-SR-BI IgG. As shown in Table 4, anti-mSR-BI IgG inhibited [³H] steroid production by 67% versus control or nonimmune IgG (P <0.0001), whereas nonimmune IgG had no significant effect versus control (P >0.2). To test antibody inhibition of steroid secretion at a lower HDL concentration, high specific activity recombinant [³H]rHDL was used. Table 4 shows that 356 anti-mSR-BI IgG inhibited [3 H] steroid production by 78% versus control or nonimmune IgG (P <0.0001) with [³H]rHDL at 5 μg protein/ml, whereas nonimmune IgG had no effect versus control (P >0.15). These data indicate that SR-BI is responsible for the delivery of most of the HDL CE to the steroidogenic pathway in Y1-BS1 adrenocortical cells.

TABLE 4
Anti-SR-B1 IgG inhibits the production of
[³H] steroid derived from [³H]HDL.
[3H] steroid, % control ± SD

| HDL | Control | Nonimmune IgG | 356 anti-SR-BI IgG |
|---|---|---|---|
| [³H]hHDL3 | 100 ± 11.3 | 90.2 ± 11 | 33.2 ± 7.1* |
| 25 μg/ml | (n = 7) | (n = 4) | (n = 4) |
| [3H]rHDL | 100 ± 3.8 | 92.9 ± 6.6 | 21.7 ± 0.8* |
| 5 μg/ml | (n = 3) | (n = 3) | (n = 3) |

*Differs from the control or nonimmune IgG, P < 0.0001.

In summary, the selective uptake of HDL CE occurs in a variety of human and other mammalian cell types and appears to be an important pathway for the movement of plasma HDL CE into the liver, as well as steroidogenic cells.

Modifications and variations of the methods and materials described herein will be obvious to those skilled in the art and are intended to be encompassed by the following claims. The teachings of the references cited herein are specifically incorporated herein.

I claim:

1. A method for modifying steroid production in a mammal comprising administering a compound altering the transfer of cholesterol or cholesteryl ester from high density lipoprotein or other lipoproteins via SR-BI to liver or steroidogenic tissues.

2. The method of claim 1 wherein the compound alters SR-BI expression in the tissue.

3. The method of claim 1 wherein the compound alters binding of SR-BI to high density lipoprotein including cholesteryl ester or other lipoproteins.

4. The method of claim 2 wherein the compound decreases SR-BI expression in the tissue.

5. The method of claim 2 wherein the compound increases SR-BI expression in the tissue.

6. The method of claim 3 wherein the compound decreases SR-BI binding to lipoprotein or transfer of cholesteryl ester in the tissue.

7. The method of claim 3 wherein the compound increases SR-BI binding to lipoprotein or transfer of cholesteryl ester in the tissue.

8. The method of claim 1 wherein the mammal is a female and the compound is administered in an amount effective to prevent normal reproductive function.

9. The method of claim 1 wherein the mammal has a disorder characterized by overproduction of steroids.

10. The method of claim 1 wherein the mammal has a disorder characterized by underproduction of steroids.

11. The method of claim 10 wherein the disorder is menopause.

12. The method of claim 1 wherein the mammal has a disorder which can be treated by decreasing production of steroids.

13. The method of claim 12 wherein the disorder is breast or prostate cancer.

14. The method of claim 12 wherein the disorder is endometriosis or fibroid tumors.

15. The method of claim 1 wherein the compound differentially alters the activity of, or expression of, SR-BI in different tissues.

16. The method of claim 11 wherein the compound increases SR-BI expression in reproductive tissues and decreases or does not increase SR-BI expression in liver.

17. A method of manufacture of a compound for use in the method of claim 1, wherein the compound is obtained by screening for binding to or alteration of activity of SR-BI in binding or transfer of cholesterol or cholesteryl ester.

18. A pharmaceutical composition for use in the method of claim 1.
